(19) United States
(12) Patent Application Publication
(10) Pub. No.: US 2006/0008512 A1
(43) Pub. Date: Jan. 12, 2006

(54) Composition and methods for improved animal performance

(76) Inventor: Danny Michael Hooge, Eagle Mountain, UT (US)

Correspondence Address: Danny M. Hooge, 8775 Cedar Pass Road, Eagle Mountain, UT, 84043, US

(21) Appl. No.: 11/178,159

(22) Filed: Jul. 7, 2005

Related U.S. Application Data
(60) Provisional application No. 60/586,104, filed on Jul. 7, 2004.
(60) Provisional application No. 60/633,081, filed on Dec. 6, 2004.

(51) Int. Cl. A61K 31/198; A23K 1/17; A23K 1/165

(52) U.S. Cl. 424/442; 514/567

(57) ABSTRACT

Disclosed is a method for converting phenylalanyl to tyrosyl residues in casein by hydroxylation (OH) and for reacting diiodotyrosine with casein to get higher thyroxine yields the thyroactive iodination of casein. A method is disclosed for elevating blood thyroid hormone levels in avian species (especially poultry species such as chickens, turkeys, ducks, quail, etc.) and other animals (e.g., mammals) by implantation, injection, or dietary (feed or water) supplementation with thyroid hormones or thyroid-active substances (e.g., L-thyroxine, triiodothyronine, defatted and dessicated thyroid, or rendered animal byproduct meal with substantial thyroid tissue), optionally in combination with other substances which improve or give additional responses, to achieve a number of economically important productive performance benefits. Manipulating thyroid hormone levels at a sensitive critical period in males to induce transient hypothyroidism results in subsequent increased testes size and sperm production. Thyroid hormones are added to fresh or diluted semen, optionally with other enhancers, to improve sperm characteristics.

### CROSS-REFERENCE TO RELATED APPLICATIONS

This application claims priority on the basis of U.S. provisional applications 60/586,104, filed Jul. 7, 2004, and 60/633,081, filed Dec. 6, 2004, which applications are hereby incorporated in their entireties by reference.

### DESCRIPTION OF THE INVENTION

As a preliminary matter, the following definitions are offered in order to provide the reader an aid in understanding the teachings of the specification. These definitions are not intended to limit the scope of the claims nor to contradict any external authority but rather are intended strictly to assist the reader in discerning the meaning of applicant's disclosure.
Animals—ruminant and monogastric animals; avian species, especially poultry (i.e., chickens, turkeys, ducks, geese, guinea fowl, pheasants, quail, ostriches, emus, and so on for meat and eggs), as well as mammals (i.e., swine, dairy cattle, beef cattle, horses, sheep, goats, rabbits, llama, alpaca, deer, elk, and so on for meat and milk) and all useful animals (e.g., companion animals, exotic pets, zoo animals and birds, fish and certain other farmed aquaculture species such as eels, and so on).
Avian species—all birds, including poultry.
Basal metabolic rate—rate of cellular metabolism (as evidenced by heat production) and its associated oxygen consumption; the major regulatory function of thyroid hormones.
Bird—any warm-blooded vertebrate of the class Aves, having a body covered with feathers, bipedal locomotion (2 legs), and forelimbs modified into wings.
Diet, feed, or ration—any composition, compound, preparation, or mixture suitable for, or intended for, consumption by animals; usually distinguished from an additive, supplement, or premix.
Iodine (iodide)—a nonmetallic, halogen element essential in nutrition, being especially abundant in the colloid of the thyroid gland (i.e., the result of “iodine trapping” by the thyroid tissue).
Molting—1) as it refers to avian species, a physiological process associated in nature with short day length and involving reduced feed intake, body weight loss, regression of reproductive organs in females (ovaries and oviduct) and reproductive quiescence in males, and feather shedding and regrowth; 2) also the name of a procedure (i.e., forced molting or induced molting) used in the commercial poultry industry to rest the birds and extend the production of table eggs or fertile hatching eggs; heretofore, usually accomplished by 6-10 hour days and feed withdrawal (fasting) or feed and water withdrawal.
Poultry—domesticated birds raised primarily for meat, egg, and/or feather production such as chickens, turkeys, ducks, geese, guinea fowl, pigeons, pheasants, quail, other game birds, ostriches, emus, swans, peafowl, and so on.
Thyroid—1) gland which produces thyroid hormones, thyroxine (T₄; containing 4 iodine atoms) and triiodothyronine (T₃; containing 3 iodine atoms); and 3) a pharmaceutical substance derived from thyroid glands obtained from domesticated animals used for food by man, the glands having been deprived of connective tissue and fat, and then cleaned, dried, and powdered for use in replacement therapy. Note that monoiodotyrosine (MIT or T₁) and diiodotyrosine (DIT or T₂) also produced in the thyroid are not hormonally active.
Thyroid function status—euthyroid is normal; hyperthyroidism indicates excess production of thyroid hormones, and “hypothyroidism” signifies under-production of thyroid hormones.
Thyroxine—a crystalline iodine-containing compound, 3,5,3′,5′-tetraiodothyronine, possessing the physiological properties of thyroid extract, used in treatment of hypothyroidism; formula is C₁₅H₁₁I₄NO₄, molecular weight is 776.8768, and iodine content is 65.3408% because iodine is relatively heavy with an atomic weight of 126.9044.
Thyroxinic—containing thyroxine, as in the term “thyroxinic substance”
Triiodothyronine—one of the compounds liberated from thyroglobulin by hydrolysis (3,3′,5-triiodothyronine); reverse T₃ is 3,3′,5′-triiodothyronine (sometimes written rT₃).
Thyroprotein or Thyroactive Iodinated Casein—thyroprotein can be produced from any tyrosine-containing proteins; as distinguished from simply iodinated casein which can contain monoiodotyrosine and diiodotyrosine (one phenyl ring with 1 or 2 iodine atoms), thyroactive iodinated casein, occasionally abbreviated herein as TIC (e.g., Protamone®, Agri-Tech, Inc., Kansas City, Mo., no longer marketed) has biphenyl ether derivatives with triiodothyronine (T₃) and thyroxine (T₄) activities by analysis such as by enzyme hydrolysis and HPLC.
Thyroactive iodinated organic compound—a categorical term for any organic compound having iodine as an integral component and having thyroid hormone activity (e.g., L-thyroxine content by HPLC analysis).

That preliminary matter now being concluded, the following description is accordingly provided.

a. Molting in the Commercial Egg Industry (Table Eggs). Induced molting of caged laying hens is crucial for the profitability of the table egg industry to extend egg production and improve shell quality (Bell, 1965; Noles, 1966; Wolford, 1984; DeCuypere and Verheyen, 1986; Kuenzel, 2003). Bell (2003) estimated that more than 75% of all commercial laying hen flocks in the U.S. are molted as part of a regular replacement program. Today, there are about 300 million caged laying hens in the U.S. However, in response to animal welfare and public relations considerations, McDonald's and Wendy's, as well as the American Veterinary Medical Association and United Egg Producers, have adopted policies designed to compel discontinuation of commonly used molting techniques that are based on feed and water withdrawal, or that cause feed avoidance. Holt (2003) stated that induced molting by the conventional feed removal (fasting) method depresses the immune system and exacerbates a Salmonella enteritidis problem.

Several low nutrient density feed molting programs have been developed recently, but cessation of egg production tends to be variable and incomplete (Biggs et al., 2004). Koch et al. (2004) reported that 4 or 8 mg melengestrol acetate (MGA), a progestin, per laying hen per day through the feed results in reversible regression of the reproductive system; perhaps 10 to 15 mg MGA daily may be required for complete cessation of egg production (0%). Szelenyi et al. (1988) induce forced molt in hens with 5 mg progesterone/day for 25 days, and feathers were shed between days 11 and 19. Johnson and Brake (1992) observed that 2,800 mg zinc/kg diet had an inhibitory action on progesterone production in F1 granulosa cells of the ovary in laying hens. Kobayashi et al. (1986) determined that zinc ion appeared to be a potent inhibitor in both T₄ and rT₃ deiodination systems in rat liver homogenates, possibly indicating a T₄ sparing effect by zinc. Burke and Attia (1994) dosed White Leghorn hens with a single i.m. injection of Lupron Depot® (Abbott Labs, N. Chicago, Ill.) formulation of leuprolide acetate, a luteinizing hormone-releasing hormone agonist, designed to release 60 mcg/kg body weight per day for 30 days and egg production dropped to 3.5% in the second week with no body weight loss. Braw-Tal et al. (2004) found a very sharp rise in corticosterone, an indicator of stress, after 2 days on molting treatments such as feed withdrawal or moderate zinc and low calcium, and 20 to 40 mg corticosterone/kg diet has been shown to cause cessation of egg production in 4 to 8 days in 98% of laying hens (Gross et al., 1983). Barron et al. (1999) deprived laying hens of light for 48 hours, followed by 8 hours of light daily, and withdrew feed from day 0 but allowed access to distilled water and oyster shell. Egg production ceased in an average of 3.2 days.

The cessation of egg production triggered by 5,000 mg iodide/kg diet is not accompanied by regression of mature ovarian follicles (although ovulation evidently ceased), and the extent of actual feather loss is minimal in young pullets whereas a typical molt response occurs in older hens (Perdamo et al., 1966; Arrington et al., 1967; Wilson et al., 1967; Herbert and Cerniglia, 1979; Albuquerque et al., 1999). The biological basis for the response of hens to 5,000 mg iodide/kg feed remains unclear.

Dramatic increases in the circulating levels of T₄ have been correlated with the normal molting process in a variety of avian species (Brake et al., 1979; DeCuypere and Verheyen, 1986; Groscolas and Leloup, 1986; Hoshino et al., 1988; and Kuenzel, 2003). Experiments have shown that feeding or injecting hens with thyroactive materials (more specifically T₄, tetraiodothyronine, rather than T₃, triiodothyronine) causes molting (feather loss) accompanied by cessation of egg production (Torrey and Homing, 1922; Zavadovsky, 1925; Cole and Hutt, 1928; Blaxter et al., 1949; Himeno and Tanabe, 1957; Verheyen et al., 1984; DeCuypere and Verheyen, 1986; Sekimoto et al., 1987; and Keshavarz and Quimby, 2002). Feeding diets containing thyroactive iodinated casein to turkeys failed to cause young (25 week old) hens to molt, but successfully induced a molt in older (yearling) turkey hens (Kosin and Wakely, 1948).

Miller et al. (1962) found when injecting 9 to 729 micrograms L-thyroxine/100 g body weight (with injections started on 3 different weeks and discontinued once the highest thyroxine level was reached, 9 mcg/100 g body weight intitially in the leg and the level tripled each week to maximum 729 mcg/100 g body weight) to White Leghorn hens 7 months of age. Excessive levels of injected thyroxine (e.g., 243 micrograms/100 g body weight) caused cessation of egg production and rapid molt, with 47% mortality, but egg weight was unaffected. Two key studies more recently clearly demonstrated that intramuscular injections of 500 to 700 μg of T₄ per kg body weight per day caused egg production to cease completely within 3 to 7 days (DeCuypere and Verheyen, 1986; Sekimoto et al., 1987). Szelenyi and Peczely (1988) treated laying hens with 0.2 or 0.4 mg thyroxine per hen for 21 consecutive days in two identical experiments and observed that: 1) the lower dose diminished egg production but did not result in molting, and 2) the higher dose stopped egg laying on the 16th day and caused a loss of contour feathers from the 14th day onward. The new plumage was completely developed in the latter group on or about the 42nd day from initial treatment.

When animals consume and digest the iodinated proteins, free T₄ (as well as T₃) is liberated and absorbed into the blood stream. For example, iodinated casein (formerly marketed as Protamone®) contained approximately 1% T₄ by weight, and provided a biologically effective source of supplemental thyroxine when fed to cows and chickens (Reineke and Turner, 1942; Irwin et al., 1943; Parker, 1943; Turner et al., 1944, 1945a, 1945b; Blaxter, 1945; Blakely and Anderson, 1948; Wheeler and Hoffman, 1948; Wheeler et al., 1948; Blaxter et al., 1949; Boone et al., 1950; Oloufa, 1955; Herbert and Brunson, 1957; Srivastava and Turner, 1967; Roberson and Trujillo, 1975; Newcomer, 1976; Harms et al., 1982; Wilson et al., 1983). Serum T₄ levels increased by >25% within two days after White Leghorn cockerels began consuming diets supplemented with 0.02 or 0.04% levels of Protamone® (Newcomer, 1976). Whether injected or administered orally, the effects of thyroactive iodinated casein were shown to be qualitatively similar to those of L-thyroxine (T₄) in poultry (Srivastava and Turner, 1967).

Turner and Reineke, Sep. 18, 1945, stated that “the administration of iodinated protein to birds in amounts substantially less than we recommend has little or no effect, while the administration of amounts substantially greater actually causes a decrease in growth and egg production”. In a trial with 2-year old laying hens, the chickens were fed thyroactive iodinated casein at levels of 0, 0.01, 0.022, or 0.04% in the diet (lots 1-4). It was observed that “hens moulted shortly after being placed in the laying batteries but the birds receiving the iodinated protein all molted at once and much more rapidly than the untreated birds. During the moult the egg production of the birds in lots 2, 3, and 4 dropped below the egg production of the controls in lot 1. However, after moulting the egg production of the hens receiving the iodinated protein rapidly passed the egg production of untreated controls. This was particularly true of birds in lots 2 and 3. The egg production of the birds in lot 3 was outstanding [0.022% level or 220 ppm].” They further stated that “preliminary tests using [a dietary supplemental level of 0.22%] iodinated protein . . . caused marked decreases in body weight of birds and [0.077%] iodinated protein . . . depressed egg production over periods of months”. The authors discussed the toxicity of thyroxine and described molting in hens resulting from consumption of excessive dietary thyroactive iodinated casein, implying that this was a danger to be avoided. They failed to realize its benefits or make any claim regarding molting in commercial flocks.

Keshavarz and Quimby (2002) evaluated the feasibility of molting 66-week-old caged laying hens with a supplement of 10 mg thyroxine/kg feed to either 96.6% corn or 91.3% grape pomace based diets, compared to traditional feed withdrawal molting. Thyroxine was added to accelerate the rate of body weight loss and to reduce the period needed to reach 30% body weight loss. A 1-day feed withdrawal, followed by grape pomace diet plus thyroxine, for inducing molt resulted in similar days to target body weight as the conventional feed withdrawal method (16 days vs 14 days, respectively) and caused similar regression of ovaries and oviduct. The 1-day fast or no fast followed by corn diet with or without thyroxine all required 28 days. The feed withdrawal control hens had 66.8% egg production from 66 to 98 weeks whereas the grape pomace diet plus thyroxine hens had 64.7% followed by corn diet plus thyroxine hens with 57.1 to 60.2%. This 10 mg thyroxine/kg of diet level was insufficient to induce a rapid cessation of egg production within 3 to 10 days, and the 1-day feed withdrawal required prior to feeding grape pomace diet plus thyroxine is now considered unfriendly with regard to animal welfare. The 10 mg/kg level of thyroxine supplementation helped reduce but did not entirely eliminate egg production, nor did it cause satisfactory regression of the reproductive tract unless coupled with feed withdrawal or substantial nutrient restriction. These researchers used 10 mg thyroxine/kg feed for its catabolic and heat production functions to hasten body weight loss, not to induce molt. They failed to make the critical discovery of optimum level needed to induce molting entirely with exogenous thyroid hormone and without feed withdrawal molting.

Therefore, L-thyroxine supplementation to complete, nutritionally well-balanced feed to induce molting is desirable. An “animal welfare friendly” molting program allowing full access to treated feed and to drinking water is beneficial for disease prevention, mortality reduction, and maintaining good relationships with egg consumers. The present invention and any inventions related thereto provide L-thyroxine as natural molting hormone and that administering a dietary level of approximately 10 to 300 mg L-thyroxine/kg (preferably about 40 mg/kg; alone on in combination with triiodothyronine as in thyroactive iodinated casein) consistently induces cessation of egg production, body weight loss, and feather molt typical of molting by feed withdrawal or natural short day length, in females of avian species. Reduced feed and calcium intake due to 40 mg thyroxine/kg diet is correctable to some extent by feeding the thyroxine treated feed on alternate days although this slows the molt induction process. Preconditioning hens with short day length (e.g., 7-10 days of 10 hours light daily), using short day length during the molt induction period, and offering low nutrient density diets with about 2% calcium facilitate the molting process. Optionally, thyroid hormone is administered in combination with supplemental magnesium, sodium salicylate, and/or protease enzyme (i.e., for improving digestibility and absorption of thyroxine). This method is suitable for commercial use.

b. Molting Other Poultry and Avian Species. Tona et al. (2002) described experiments molting commercial Cobb broiler breeder hens, 55 to 62 weeks of age. Molting increased egg internal quality (Haugh units) and hatchability of eggs compared to unmolted controls. Herremans (1988) reported from molting studies with white- and brown-egg layers and with broiler breeder hens that “at comparable age the moulting response was considerably more extensive in broiler-breeders than in layers”. However, Hemken (1981) stated that adding iodine at 50 mg/kg to breeder hen diets caused a reduction in hatchability of eggs. Therefore, hatchability of fertile eggs from hens during T₄ molting treatment is monitored for iodine content, and these may have to be diverted to other uses such as human consumption (150 mcg/egg maximum) or rendering.

Bilezikian et al. (1980) found that 3 mcg L-thyroxine/mL water (600 to 900 mcg/bird/day) to 20 to 25 week old turkey females caused hens to rarely lay eggs and shells were incompletely calcified; however, hypothyroid turkeys did not lay eggs either. Based on previous work by Lien and Siopes (1989) indicating that T₄ may be involved with photorefractoriness (insensitivity to light), Lien and Siopes (1993) dosed laying turkeys with 0.075 to 2 mg L-thyroxine/bird/day by intramuscular injection for either 2 or 3 weeks following 10 weeks of photostimulation to determine photorefractoriness, feed consumption, and degree of molting. Turkey hens in two trials were 40 and 72 weeks of age, respectively. Transient depressions in egg production and molting were observed during and after T₄ treatments. Feed consumption declined with increasing T₄ doses. Turkeys in the 2 mg L-thyroxine/hen/day treatment terminated egg production during T₄ treatment and remained out of production for 4 weeks after treatment. These turkeys treated for 3 weeks molted body feathers and most primary remiges. Thyroxine administration did not result in photorefractoriness (as in starlings and coturnix quail) because turkey hens came back into egg production. Injecting large numbers of turkey hens or adult females of other poultry species is economically infeasible due to the exorbitant labor expense. Pairs of Humboldt penguins at Tokyo Sea Life Park were reported by Otsuka et al. (1998) to molt around the end of July or early August (males usually started earlier), coincident with a sharp increase in plasma T₄ which doubled within 10 days and lasted for a month. Duration of feather molting was short, averaging about 13 days.

According to the present invention, L-thyroxine or thyroxine-containing substance is administered to adult females of avian species, preferably via the diet at approximately 40 mg/kg feed (11 to 300 mg/kg) to induce molting and extend egg production.

c. Control of Pest Bird Populations. The detrimental effects of pigeon excreta and feathers on barns, city buildings, and sidewalks are well known. Wild birds of various species damage agricultural crops, aircrafts, and other property, as well as spread disease. Since 1960, 22 military and civilian planes have been destroyed by bird strikes, as bird-aircraft collisions are called. In 2002, some 9,900 bird strikes caused $499 million in damages, and in 2003 three small plane-bird collisions killed five people (Ault, 2004). Nicarbazin® (Koffolk, Inc., Rancho Santa Fe, California; 125 mg/kg of diet), a poultry coccidiostat, has been researched in food baits for pigeons and wild birds to decrease hatchability by altering yolk membrane permeability of fertile eggs so that albumen and yolk commingle creating an unfavorable condition for the early stage embryo (“goose contraceptive”; www.aphis.usda.gov/ws/nwrc/nicarbaz.htm). Hughes et al. (1991) dosed broiler breeder hen feeds with 0, 25, 50, or 100 mg Nicarbazin® and found a reduction in egg weight with 100 mg/kg in 6 days, no influence on fertility of eggs, but reductions in hatchability at 50 or 10 mg/kg within 5 or 6 days (31% hatchability with 50 mg/kg on days 11 and 12).

Cook et al. (Apr. 2, 1996; U.S. Pat. No. 5,504,114) disclosed the use of an effective amount (e.g., 0.5% level) of conjugated linoleic acid in the diets of female adult birds to prevent eggs from hatching by decreasing the total unsaturated fatty acid content of the fertilized egg yolk. The conjugated linoleic acid is selected from 9,11-octadecadienoic acid, 10,12-octadecadienoic acid, or mixtures or non-toxic salts thereof. Among the most common bird pests that need to be controlled are seagulls, pigeons, blackbirds, grackles (see also Warren, 2005), starlings, crows, sparrows, and waterfowl.

These and a number of other methods such as timed booms, poisons, shooting, anthocyanin bird repellent for seeds, and so on have been minimally effective or have not received widespread acceptance. Therefore, according to the present invention sufficient thyroid product containing T₃ and T₄ such as iodinated casein or rendered thyroid tissue is administered (e.g., L-thyroxine content 10 to 300 mg/kg diet; preferably 40 mg L-thyroxine/kg) to induce molting in adult males and females or L-thyroxine alone for females only (10 to 300 mg/kg diet; preferably 40 mg/kg) as males have a lower tolerance for L-thyroxine administered singly, or in combination with Nicarbazin® (a broiler chicken chemical coccidiostat), and/or conjugated linoleic acid to impair hatchability of any fertile eggs that are laid. This approach is a non-lethal method to address the problem of wild pest birds in order to maintain them at appropriate population levels.

d. Conventional Methods of Making Thyroactive Iodinated Casein or Levothyroxine. In the manufacture of thyroactive iodinated casein, although casein has on average about 5.0% tyrosine which could theoretically yield about 9.38% thyroxine, it actually yields about 1% on analysis. This calculation is based on the statement of Reineke and Turner (1945) that casein with 5.65% tyrosine (slightly high estimate) would have theoretical yield of 10.6% thyroxine.

IG Farbenindustrie AG (Patent No. GB492265, Sep. 13, 1938; Manufacture of Thyroxin), described manufacture of thyroxine from iodinated proteins by a hydrolytic decomposition, with the iodination carried out in weakly alkaline aqueous solution at moderately raised temperature by gradually adding finely pulverized iodine and stirring with a metal rod as catalyst, hydrolyzing the iodinated protein, and purifying the product. The Million test used for residual iodine contains mercury and is environmentally unfriendly.

Quaker Oats Co. and American Dairies Inc. (GB568183, Mar. 22, 1945, Thyroprotein Com-position and Method of Making the Same; GB598679, Feb. 24, 1948, Improvements Rel-ating to Processes for the Production of Thyroxine; GB598680, Feb. 24, 1948, Thyroprotein Composition and Method of Making the Same) detailed a method for manufacture of thyroprotein and improvements relating thereto. GB568183 included a mixture of iodine and potassium iodide in aqueous solution. In GB598679, L-thyroxine was obtained from thyroprotein compositions without racemization by hydrolyzing (refluxing together) in an aqeuous solution of an acid and N-butyl alcohol and extracting substantially pure thyroxine. The acid may be a mineral acid such as hydrochloric acid, but preferably sulfuric acid. Patent GB598680 iodinated protein at 50 to 70° C. in an aqeous solution having a pH of 6.8 to 10 until a negative Million test, then at 50 to 100° C. for 12 to 72 hours with aeration, vigorous stirring, and in the presence of metal or peroxide catalyts. Increasing increments of iodine to protein were tested in relation to thyroxine output.

U.S. Pat. No. 2,329,445 (Turner and Reineke, Sep. 14, 1945) described Thyroprotein and Method for Making the Same. Skim milk could be replaced by: casein, milk albumin; blood serum, albumin, or globulin, egg albumin, meat meal or its protein, or other animal proteins; cottonseed meal, gluten meal, soybean meal, peanut meal, coconut meal or other high protein ingredients with low oil contents. Molecular iodine is preferred, but it can be replaced by salts of iodine such as NaI, KI, NaIO₃, or others capable of releasing free iodine. This and similar processes such as chlorination and bromination are well known in the art.

Turner and Reineke (Jul. 3, 1945), in U.S. Pat. No. 2,379,842, Thyroprotein Composition and Method of Making the Same, stated that to obtain maximum thyroxine activity, only sufficient iodine is added to substitute 2 atoms of iodine per molecule of tyrosine (i.e., 4 to 6 atoms of iodine per molecule of tyrosine). Excess iodine next iodinates the imidazole ring of histidine, and then oxidizes tryptophan and part of the sulfur of the cysteine complex (cystine). The iodination of tyrosine proceeds by substitution according to the equation: Tyrosine+2I₂=diiodotyrosine+2 HI.

1. Improvement 1. Greater Thyroxine Yield by Converting Phenylalanyl to Tyrosyl Residues. Theoretically, in proteins or peptides the glycyl residues are convertable into alanyl residues which are in turn convertable into phenyalanyl residues based on published reactions carried out with the respective amino acids (Singh et al., 1987). Surprisingly, it is possible to increase thyroxine content of thyroactive iodinated casein by first converting some or all of the 4.5% phenylalanyl residues present to tyrosyl residues, which differ in structure by only one hydroxyl (OH) group and such OH group can be added to each of the phenylalanyl residues by hydroxylation. After making this modification to casein, there are 5.0% original tyrosyl residues plus up to 4.5% phenylalanyl residues converted to tyrosyl residues, amounting to 9.5% total tyrosyl residues with a theoretical thyroxine yield of 17.8%.

According to the present invention, phenylalanyl residues are converted to tyrosyl residues needed in the manufacture of thyroactive iodinated casein (or other proteins or peptides). Dakin and Herter (1907) showed that hydrogen peroxide oxidizes phenylalanine in both the side chain and nucleus. Raper (1932) used phenylalanine, FeSO₄.7H₂O, and hydrogen peroxide in a 4-day reaction to form tyrosine. From 4 g of phenylalanine, relatively low yields of 167 mg crude and 43 mg of first-crop recrystalline tyrosine were obtained.

Hydroxylation of phenylalanine in the presence of cysteine was reported by Gerthsen (1962). Guroff and and Rhoads (1969) researched the hydroxylation of phenylalanine by Pseudomonas species, and Friedrich and Schlegel (1972) used Hydrogenomonas eutropha H 16. Hydroxylation of phenylalanine by the hypoxanthine-xanthine oxidase system is possible (Ishimitsu et al., 1984). Ishimitsu et al. (1990) reported that phenylalanine can be converted to p-, m-, and o-tyrosine by irradiation with ultraviolet light through the decomposition of water (H₂O), and the reaction is facilitated by riboflavin (Ishimitsu et al., 1985).

Phenylanine hydroxylase in liver enzyme catalyzes the catabolism of excess phenylalanine in the diet to tyrosine; the vast majority of cases of phenylketonuria (PKU) are due to deficiencies of this enzyme. The reaction which catalyzes the conversion of phenylalanine to tyrosine involves oxidation of NADH to form NAD+ and the reduction of O₂ to form H₂O. It is also dependent on conversion of tetrahydrobiopterin into dihydrobiopterin which serves to carry the electrons from NADH to O₂ (www.bio.davidson.edu, accessed Mar. 25, 2005). Knight (1982) experimented with the NADPH-dependent tyrosyl-peptide iodinating activity of porcine thyroid tissue.

Fitzpatrick (2003) described the proposed mechanisms of the aromatic acid hydroxylation. He stated that phenylalanine hydroxylase ortholog is present in at least 17 different bacterial genomes, although not in Escherichia coli; however, only the enzyme from Chromobacterium violaceum has been characterized to any extent. The eukaryotic enzymes are all homotetramers. The pterin dependent hydroxylases are iron dependent enzymes requiring one iron atom per subunit for activity. When isolated, the iron is typically in the ferric state whereas the active form is ferrous. Tetrahydropterins readily reduce the ferric iron in phenylalanine hydroxylase in vitro suggesting that they are the physiological reductant. These enzymes begin and end each catalytic cycle in the ferrous form. All three substrates (phenylalanine, dimethyltetrahydropterin as a ligand for iron in ferrous state, and oxygen) must be bound before catalysis occurs. Triaminopyrimidines will function in place of tetrahydropterins as substrates for the enzymes because most reactions involve the pyrimidine ring of the pterin. Site directed mutagenesis revealed that mutant D425V tyrosine hydroxylase shows a preference for phenylalanine over tyrosine of 8,000-fold. Regarding oxygen activation by pterin during catalysis, Chromobacterium violaceum forms hydrogen peroxide (H₂O₂) in the presence of phenylalanine if the metal-free enzyme is used. Oxygen addition to the amino acid substrate is consistent with the presence of an electrophilic hydroxylating intermediate such as Fe(IV)O and with the observation that these enzymes exhibit NIH shifts, 1,2 shifts of the substituent at the site of hydroxylation to the adjacent ring carbon (4-hydroxylation). Anonymous (2002) stated that the NIH shift, first observed in the enzymatic hydroxylation of phenylalanine, was one of the landmark findings in organic chemistry.

In methods according to the present invention, current knowledge in the fields of biochemistry, microbiology, and biotechnology is employed to create microbes with enhanced production of hydroxylase to commercially hydroxylate phenylalanyl residues into tyrosyl residues in proteins and/or peptides. Tyrosyl residues are then iodinated to iodothyronines (e.g., thyroactive iodinated casein) according to established procedures in the prior art or an improved process such as the following.

2. Improvement 2. Sparing Tyrosyl Residues in Protein Using Other Phenyl Sources. Tyrosyl residues in the casein must receive a second phenyl ring (each ring containing 2 iodine molecules), known as a coupling reaction, to form thyroxine (i.e., biphenyl ether derivative of tyrosine). With casein as the starting material, the transferred phenyl ring comes from another tyrosyl residue within the protein thus creating an alanyl residue which cannot be iodinated.

Coe (Jun. 29, 1999; U.S. Pat. No. 5,917,087) described improvements to a six-stage process for production of sodium L-thyroxine from L-tyrosine originally developed by Ginger et al. (June, 1959; U.S. Pat. No. 2,889,363) and Anthony et al. (June, 1959; U.S. Pat. No. 2,889,364). The newer process involves: 1) oxidative coupling of an diiodo-L-tyrosine to form a biphenyl ether derivative, 2) catalyzed by a manganese salt, 3) performed at a pressure of about 20 atmospheres, 4) in the presence of an organic amine additive, 5) using a gaseous oxidant comprised of oxygen and an inert diluent, 6) acid hydrolysis of the biphenyl ether derivative with HCl to L-thyroxine HCl salt, and 7) generation of sodium L-thyroxine from the L-thyroxine HCl salt.

Achieving higher thyroxine yield is accomplished according to the present invention by providing additional sources of phenyl groups such as: 1) hydrolyzed or partially hydrolyzed casein providing free tyrosine or peptides such as dipeptides containing tyrosine, 2) diiodotyrosine, and/or 3). 4-hydroxy-3,5-diiodophenylpyruvic acid (p-hydroxy-3,5diiodophenylpyruvic acid) (Blasi et al., 1969). Block (1940) confimed that the conversion of diiodotyrosine into thyroxine was possible in vitro using a completely synthetic diiodotyrosine as starting material. Van Bruggen and West (May 31, 1955; U.S. Pat. No. 2,709,671) proposed using freshly activated (e.g., with hydrogen sulfide) papain as the enzyme to hydrolyze casein to be the starting material for making “thyroprotein”, more specifically iodinated hydrolyzed casein. In the present invention, half of the starting material is hydrolyzed casein (e.g., dipeptides) and half is casein. The casein dipeptides containing tyrosyl residues, being somewhat more chemically reactive than native protein, can more readily interact with the casein tyrosyl residues than the intact tyrosyl residues can interact with each other. Casein peptide tyrosyl residues provide the necessary second phenyl group to form thyronines (tri- or tetra-iodination as T₃ or T₄, respectively) at the casein tyrosyl residues sparing some of the casein tyrosyl from loss of a phenyl ring and therefore able to continue to form additional thyronines producing higher yields.

According to the present invention, portions of the different processes using either casein or L-tyrosine as starting materials are combined so that casein having intact tyrosyl residues are reacted by phenolic coupling with casein short-chain peptides, free diiodo-L-tyrosine, 4-hydroxy-3,5-diiodophenylpyruvic acid, and/or other phenyl group containing molecules to obtain a higher yield of thyroxine activity in the thyroactive iodinated casein product, a well known “animal drug” product. It is important because of historical use of iodinated casein as an animal feed ingredient that casein be the main starting raw material.

For example, as in the Coe patent, iodination of L-tyrosine to 3,5-diiodo-L-tyrosine is done first, followed by removal of any excess iodine. Then suitable protecting groups are attached to the iodinated tyrosine for the amine group (e.g., acetylation with acetic anhydride and a base) and for the carboxy group (e.g., esterification with ethanol in sulfuric acid). Separately, casein which is put through the initial iodination of tyrosyl residues (2 iodine atoms per tyrosyl residue) and then added to the Coe process vessel. Oxidative coupling is accomplished under pressure of 20 atmospheres with MnSO₄ and H₃BO₄ in ethanol with a piperidine additive. Alternative manufacturing processes to promote oxidative coupling of the two compounds, casein and diiodotyrosine, are acceptable. In analagous procedures, casein is reacted with hydrolyzed casein or with the diiodophenylpyruvic acid to increase thyroxine yields.

e. Proprietary Rendered High Thyroid Tissue Content Animal Byproduct Meal. Armour™ Thyroid tablets (USP) sold by Forest Laboratories, Inc., St. Louis, Mo. contain defatted, desiccated porcine thyroid tissue, and a 60 mg tablet is approximately equivalent to 100 mcg levothyroxine (T₄). The tablets contain triiodothyronine (T₃) and thyroxine (T₄) in about a 1:4.22 ratio. Except for very limited veterinary use in pets and livestock, the defatted and desiccated porcine thyroid product has not found application in commercial animal agriculture but is sold as an FDA regulated human drug product (medication). Today, thyroxine varies in cost according to the source, from levothyroxine ($40/g thyroxine) to thyroactive iodinated casein ($1.25-2.50/g thyroxine). Development of proprietary rendered animal byproduct meals containing substantial amounts of thyroid tissue (e.g., 1 g thyroxine per 600 g defatted, desiccated porcine thyroid) may reduce the cost, encourage supplementation in animal feeds, and improving profitability.

For a company to utilize a feed ingredient in the U.S., the Association of American Feed [State] Feed Control Officials (AAFCO, 2003) must first approve a definition for it to be included on the feed label (tag). AAFCO does not have any ingredient definition that specifically mentions thyroid tissue, except in a general way in 9.65 Glandular Meal and Extracted Glandular Meal “obtained by drying liver and other glandular tissues from slaughtered mammals”. Note that neither rendering (defatting) nor poultry are mentioned. Ingredient definition 9.42 for Animal By-Product Meal is the rendered product from animal tissues, exclusive of any added hair, hoof, horn, hide trimmings, manure, stomach and rumen contents, except in such amounts as may occur unavoidably in good processing practices. In some other definitions, either “mammals” or “poultry” are specified as the source of raw materials, with the word “animal” generally being used as the combined species term. For example, ingredient 9.68 Animal Digest “shall be exclusive of hair, horns, teeth, hooves, and feathers . . . ” indicating that it encompasses both mammals and poultry. The Animal By-Product Meal is therefore ambiguous, but may probably include either mammal (beef and swine) or poultry tissues under the term Animal. It is not intended as a “catch all” category that allows blending of diverse animal protein supplements.

According to the present invention, thyroid tissue is removed from beef cattle, pig, broiler chicken, and turkey carcasses during processing, maintained segregated or with other appropriate tissues, and rendered separately or with other tissues to create suitable products with high thyroid tissue content (e.g., 1% to 100%, preferably 50% to 95%) and natural thyroid hormone activity. This may satisfy both Food and Drug Administration and AAFCO requirements for use of Animal By-Product Meal legally in feeds of poultry, livestock, and all useful animals including zoo animals and exotic pets. The product is standardized with regard to iodine content and iodothyronines profile (i.e., by hyrdolysis and HPLC) in the typical or guaranteed analysis and no animal performance claims are made regarding it. Other AAFCO definitions may be approved in the future which would allow thyroid tissue to be used in different products. Optionally, magnesium is added or other supplementation to the rendered product is done.

f. Increased Need for Magnesium During Administration of Exogenous Thyroid Hormones. Because thyroid hormones stimulate metabolic activity (basal metabolic rate), supplementing treated birds or other animals with magnesium at levels approximating 5% to 300% of the dietary minimum requirement level is disclosed in this invention to accommodate the accelerated tissue metabolic rate (magnesium-requiring enzymes in oxidative phosphorylation), to help maintain normal blood magnesium levels, and yet to have minimal adverse effects (e.g., hypocalcemia and decreased blood potassium). Only about 1% of the body magnesium is found in blood. Magnesium is administered long with L-thyroxine or thyroxine-containing substance, despite a range of existing dietary magnesium levels, to assure magnesium adequacy during thyroxine administration because excess magnesium is safely eliminated from the body.

Voisin (2005), in his book Grass Tetany published online, stated that it is quite remarkable that thyrotoxicosis and marked magnesium deficiency give rise to similar symptoms (i.e., vasodilation, hyperirritability of the nervous system, cardiac irregularity, increased calcium excretion in the urine, loss of body weight, and finally fever), and conversely similarities are claimed between hypothyroidism (with myxoedema) and certain effects of excess magnesium. Surprisingly, these observations of a general nature indicate the existence of a close physiological relationship between the metabolism of thyroxine and that of magnesium. This relationship was confirmed in research with young rats (Vitale et al., 1957). In a study using purified diets with 200 to 1,600 mg magnesium/kg, the rats fed 10 mg L-thyroxine/kg diet gained 60 g in 16 days when the diet had 400 mg magnesium/kg. However, when rats were fed 4 mg L-thyroxine/kg diet, they required 1,600 mg magnesium/kg diet (400% of 400 mg/kg level) to attain the same weight gain. The 200 mg magnesium/kg and 2 mg L-thyroxine/kg diet diminished considerably the magnesium content of the blood serum which fell from 1.87 to 0.50 mg %. In rats fed the thyroxine diet with high magnesium (1,600 mg/kg), the serum magnesium returned more or less to normal (1.67 mg %). It was concluded that in conditions of hyperthyroidism, more magnesium is consumed in the tissues. As a result of activation of the thyroid, it can happen that the body is no longer able to meet the requirement for increased consumption of magnesium, therefore a state of hyperthyroidism favors hypomagnesiumaeia. Thyroid hormones are involved in energy production resulting in increased oxygen consumption in tissues, and magnesium is an essential part of respiratory enzyme systems (Gershoff et al., 1958).

Low ambient temperatures (cold atmospheres) increase magnesium requirements of animals. Young rats kept at 25° C. (77° F.) required 40 mg dietary magnesium/kg to obtain 50 g body weight in 24 days whereas at 13° C. (50° F.) they needed 160 mg magnesium/kg of diet (400% of 40 mg/kg level). In other rats it has been shown that a “syndrome of adaptation” occurs after about 40 days at which time thyroid output returns to normal (Voisin, 2005).

Vitale et al. (1957) stated that serum magnesium is lower in thyrotoxic human patients, and upon treatment, serum magnesium levels return to normal. Thyrotoxic patients may require higher intakes of magnesium than normal to attain magnesium balance. Wuttke and Kessler (1976) suggested that serum magnesium concentration is primarily determined by L-triiodothyronine (T₃). Oliver (1978) administered L-thyroxine at 25 mg/dg of body weight subcutaneously for 30 days to hyperthyroid rats along with 25 mg of magnesium sulfate/dg of body weight, and there was increased magnesium concentration in most tissues. Magnesium increased in tissues of hypothyroid rats given magnesium sulfate as well. Simsek et al. (1997) reported that in an L-thyroxine-induced hyperthyroidism condition, experimental animals showed a significant decrease in erythrocyte calcium, magnesium, and zinc concentrations, and a significant decrease in plasma magnesium concentration suggesting that homeostasis of calcium, magnesium, and zinc is altered. Monson (1963) indicated that hyperirritability is associated with a fall in the serum magnesium level in rats, dogs, and rabbits, and that the critical serum magnesium level appears to be of the order of 1.0 mg/100 mL.

Taurine by its cell membrane-stabilizing, Ca-binding, and cGMP level-lowering effects and possibly through a specific action as a “Mg-sparing” parathyroid hormone (i.e., gamma-L-glutamyl taurine) appears to be important in regulation of magnesium homeostasis (Durlach and Durlach, 1984). Taurine lowers elevated blood pressure, retards cholesterol-induced atherogenesis, prevents arrythmias, and stabilizes platelets—effects parallel to those of magnesium (McCarty, 1996). Magnesium taurate contains both magnesium and taurine components.

According to the present invention, supplementing diets of poultry or other avian species as well as dairy cattle, dairy goats, and sows in lactation (which are susceptible to neuromuscular disorder “grass tetany” due to magnesium deficiency), and other classes of animals (including humans), with thyroid hormones plus magnesium, taurine (e.g., about 0.025-0.15% of diet), or both, improves magnesium homoeostasis inasmuch as thyroid tissue and function and magnesium metabolism are similar across species. In dairy cattle, MgO has been fed at 54 and 108 g/head/day depending on the needs of the cow to raise serum Mg, and Epsom salt has been fed at 85-90 g/head/day.

g. Sodium Salicylate Potentiates Thyroxine by Increasing Free Dialyzable Form. In this invention, coadministration of thyroxine with aspirin or its first metabolite sodium salicylate potentiates the thyroid hormone. Hoch (1965) reported a synergism between effects of L-thyroxine and sodium salicylate in euthyroid rats. Musa et al. (1968) studied effects of salicylates on the distribution and early plasma disappearance of thyroxine in man. Langer et al. (1977) measured the disappearance of loading doses of thyroxine (100-20,000 microg T₄ i.v. per rat weighing about 400 g) using frequent blood sampling with maintenance of isovolemia in anaesthetized animals and demonstrated that bound T₄ was displaced from plasma proteins by sodium salicylate. Langer et al. (1981) observed after sodium salicylate (200 mg/kg body weight) was injected i.v. into male rats, and blood samples taken 30-240 minutes later, that there occurred an immediate 20% decrease in plasma T₄ level along with a 60% decrease in T₃ and a 20% increase in reverse T₃.

Goussis and Theodoropoulos (1990) used serum from healthy volunteers and adjusted it to 0 or 10 mM sodium salicylate. The free dialyzable fraction of T₄ in vitro was raised by 125% after addition of sodium salicylate. However, the % of total T₄ termed bioavailable T₄ transported into the liver of rats on one pass was not significantly different in control or sodium salicylate treated animals. Sodium salicylate inhibits blood thyroxine binding to transthyretin causing a rapid increase in circulating free T₄ which decreases the activity of the enzyme converting T₄ to T₃. Chopra et al. (1980) described the inhibition of hepatic outer ring monodeiodination of thyroxine and 3,3′,5′-triiodothyronine by sodium salicylate.

XiaoTing and YouMing (2002) showed that 500 or 1,000 mg aspirin/kg diet fed to 42 week old White Leghorn hens for 10 weeks during heat stress decreased serum T₃ and T₄, but increased egg production and shell thickness and decreased feed conversion ratio, compared to control diet.

According to the present invention, exogenous thyroid hormones are potentiated with aspirin-related compounds that increase the free T₄ in blood by inhibiting binding to proteins to accomplish desired effects in food-producing animals (e.g., 0.9 to 1.75 fl. oz sodium salicylate containing 460 g/quart per 1,000 lb of body weight of birds or animals). Preferably, L-thyroxine (T₄) is administered with sodium salicylate because it reduces deiodination of T₄ to T₃. Sodium salicylate is administered with T₄ and T₃ in cases when retaining more T₄ is desirable, such as in molting.

h. Enzyme(s) to Hydrolyze Thyroprotein for Better Digestibility and Thyroid Hormone Release. Thyroactive iodinated casein has about 60% digestibility in chickens. According to the present invention, exogenous enzymes such as proteases (which hydrolyze peptide bonds in proteins) are administered to better digest the thyroprotein (e.g., casein) and release more of the thyroxine component. Barendse et al. in U.S. Pat. No. 6,500,426 approved Dec. 31, 2002 stated that proteases are sometimes designated as peptidases, proteinases, peptide hydrolases, or proteolytic enzymes. Protease may be of the exo-type that hydrolyses peptides starting at either end thereof, or of the endo-type that act internally in polypeptide chains (endopeptidases).

i. Coating for Thermostability and/or Altering Physical Characteristics for Better Handling. For thyroid hormone(s) within a feed supplement to retain activity after steam pelleting at temperatures to around 190° F., some special coating or protective material must be applied to it. Feeds are usually made in either mash (simply ground and mixed) or pelleted form. According to the present invention, a feed ingredient containing thyroid hormone(s) is processed so as to make it thermostable to pelleting, to form granules, micro-granules, or other physical forms to prevent dustiness, enhance flowability, improve distribution within a batch of feed during mixing (i.e., uniform particle size improves coefficient of variation in mixing), add color, impart flavor, and so on by technology known in the industry. Barendse et al. in U.S. Pat. No. 6,500,426 approved Dec. 31, 2002 taught that the granules (for enzymes in their case) can contain a carrier such as an edible carbohydrate polymer, and one or more hydrophobic, gel-forming, or water-insoluble compounds such as cellulose, polyvinyl alcohol (PVA), or an edible oil. The carrier is starch obtained from corn, potato, rice or other plant sources such as tapioca, cassaya, wheat, maize, sago, rye, oat, barley, yam, sorghum, or arrowroot. The cellulose can be derivatized and consist of hydroxy-propyl-methyl-cellulose, carboxy-methyl-cellulose, or hydroxy-ethyl-cellulose. The edible oil is canola oil or soy oil. The granule can contain a high melting wax or fats which also serve as matrix material or coating sometimes containing a second active ingredient. If necessary, screening is done to further improve uniformity of particle size. Granules obtained can be subjected to rounding off (e.g., spheronisation), such as in a spheromiser (e.g., a Maurmeriser™ machine) and/or compaction.

Mitra et al. (May 2, 2000; U.S. Pat. No. 6,056,975) disclosed a low moisture (<4.5%) preparation including an inorganic salt, carbohydrate (molecular weight >500), and glycine to stabilize levothyroxine sodium. Patel et al. (2003) stated that levothyroxine tablets, 50 micrograms each, have been marketed to humans for many decades (since about 1955) but have had numerous recalls due to degradation and failure to meet potency. They reported that the stability of aqueous slurries was improved as the pH of the slurry was increased from pH 3 to 11. Levo-thyroxine manufactured with 88.9% of dibasic calcium phosphate (“dicalcium phosphate”) and 10% of a basic pH modifier such as sodium carbonate, sodium bicarbonate, or magnesium oxide met the USP assay requirements at both 3 and 6 months of storage.

j. Thyroactive Iodinated Organic Compounds to Prevent Iodine Deficiency. Hoffmann La Roche (GB918409; Feb. 13, 1963; Animal Feeds) patented a specific growth-promoting supplement for animal feed which included ubiquinones (1 to 100 mg/kg) as antioxidants, soybean meal, DL-methionine, sucrose, zein, choline chloride, iodinated casein (1% thyroxine), a specified salt mixture, and a specified vitamin mixture. Thaxton (May 17, 1994; U.S. Pat. No. 5,311,841) disclosed a method for the delivery of vaccine or other medicants, including thyroxine, via injection into the yolk sac of newly hatched poultry. The substance is then released into the hatchling's system as the yolk is absorbed within days. May (1980) reported that dietary L-thyroxine at 1 mg/kg of diet in two experiments increased serum thyroxine levels (88.7 vs 41.1 ng/mL; 69.3 vs 21.1 ng/mL) of broiler chickens, 0 to 28 days of age. Using a level of 0.1 mg L-thyroxine/kg of diet gave serum thyroxine levels essentially equal to control birds. Bilezikian et al. (1980) put 3 mcg L-thyroxine/mL of drinking water of female turkey 20-25 weeks of age (12 to 15 lb) to induce hyperthyroidism, and intake was about 600 to 900 mcg/bird/day. Schone et al. (1997) stated that for diagnosis of the iodine supply status, the iodine concentration of sows milk should be analyzed, and the lower limit is defined as 50 mcg/L milk based on 5 random samples per sow. Serum iodine and T₄ levels are not suitable criteria because they remain moderate in deficiency.

According to the present invention, exogenous thyroid hormones are administered to partially or completely replace inorganic sources of the essential nutrient iodine for the purpose of providing more biologically active substances. For example, thyroxine has 65.34% iodine.

k. Overcoming Effects of Antithyroid or Goitrogenic Substances in Feed Ingredients. High calcium diets have been shown to be goitrogenic (Sampson and Putzki, 1952). Hemken (1981) wrote that 2% or more dietary calcium carbonate (limestone) increases the need for iodine. Clandinin (1989) stated that the enzyme myrosinase which converts progitrin to goitrin, the principal goitrogenic factor in rapeseed meal, causes minor thyroid enlargement and slightly reduced transfer of iodine to eggs. Summers and Leeson (1977) showed that thyroid weight of male White Leghorn chicks could be decreased from 7.8 mg/100 g body weight with no supplemental thyroxine to 5.8 and 5.8 mg/100 g body weight using 0.05 or 0.10% dietary iodinated casein with 1% thyroxine activity. Chicks fed a diet with rapeseed meal containing goitrogens had thyroid weight of 8.5 mg/100 g body weight. Roos and Clandinin (1975) reported that less ¹²⁵I was transferred into the eggs of hens fed diets containing rapeseed meal and a source of myrosinase to liberate the antithyroid compounds from the glucosinolates.

Spiegel et al. (1993) studied growing pigs feed diets containing rapeseed meal (15%) with relatively high levels of glucosinolates and goitrin had significantly lower serum free thyroxine levels compared to soybean meal based controls and developed hypothyroidism. Thyroxine supplementation kept serum free thyroxine levels normal. Schone et al. (1997) fed sows during late pregnancy and lactation diets containing 2.5% rapeseed meal that supplied 10 mM glucosin-olates/kg of diet and found decreased milk iodine concentration and serum thyroxine in pigs, and a tendency toward lower (8%) litter weight at weaning compared to control group.

Lin et al. (1990) administered daily doses of gossypol acetic acid, at levels of 0, 1, 5, or 10 mg/kg body weight in 0.5 mL potassium phosphate buffer from 34 to 49 days of age to female Sprague-Dawley rats. Fifteen days after gossypol treatment at 5 or 10 mg/kg/day, significant decreases were found in the concentrations of free T₄ (21.96 and 11.12 vs 55.90 mg/mL for control), the T₃ (430 and 359 vs 670 pg/mL for control), and the reverse T₃ (37.20 and 24.20 vs 71.7 pg/mL for control). Gossypol exerted its antithyroid function by an unknown mechanism that triggered an interference in body metabolism causing the loss of food intake and body weight gain in young female animals.

Divi et al. (1997) noted from rodent research with soy products, widely used in human infant formulas and vegetarian diets, that the acidic methanolic extract of soybeans contains the isoflavones genistein and dadzein which induce goiter and thyroid neoplasia in the animals.

According to the present invention, to help relieve the problem of specific antithyroid and goitrogenic compounds in feeds or to allow expanded use of ingredients containing such substances, exogenous thyroid hormones are administered alone or in combination with iodolactones, iodoaldehydes, or iodide plus docosahexaenoic acid (DHA) to minimize thyroid enlargement (goiter) and improve productive performance.

1. Iodolactones and/or Iodoaldehydes to Correct or Inhibit Thyroid Enlargement (Goiter). Within the past decades, multiple iodolipid classes have been identified in thyroid tissue. Thyroid cells are able to iodinate polyunsaturated fatty acids, with alpha-iodohexadecanal (alpha-IHDA) as the major compound of an iodolipid fraction. It exerts multiple inhibitory effects on adenylate cyclase, NADPH-oxidase, and thyroid peroxidase. Delta-iodolactone (ring structured derivative of unsaturated fatty acid) has been identified in human thyroid tissue, and this compound seems to act as a mediator of iodine in thyroid cell growth regulation, especially in the autoregulation of cAMP-independent thyroid cell proliferation (Dugrillon, 1996). Langer et al. (2003) stated that delta-iodolactone in very low concentrations—comparable to iodide in higher concentrations—not only inhibits growth but also induces very rapid apoptosis without necrosis in intact thyroid follicles. Stimulation of porcine thyroid follicles in vitro with 2 and 20 microM iodide rapidly induced a rate of apoptosis (4-6%) comparable to about 40-fold lower doses of delta-iodolactone (0.05 to 0.5 microM). This study may explain why iodine supplementation even in high doses does not lead to thyroid atrophy but only to normalization of thyroid size. Panneels et al. (1996) stated that 2-iodohexadecanal (2-IHDA) is a major thyroid iodolipid that mimics the main regulatory effects of iodide on thyroid metabolism. Chazenbalk et al. (1985) found a highly significant correlation between iodination of lipid and of protein (r=0.906) in calf thyroid slices, suggesting that both reactions may be related. Dugrillon and Gartner (1992) observed that treatment of isolated porcine thyroid follicles with docosahexaenoic acid (DHA, C22:6, n3) at 100 and 300 microM concentrations significantly enhanced the inhibitory effect of 10 microM of iodide on thyroid follicle proliferation (45±4% versus 84±2%). According to the present invention, the problem of excess thyroid colloid tissue production (goiter) is corrected by use of iodolactones and/or iodoaldehydes with thyroid hormomes or by DHA and iodide.

m. Improving Growth and Feed Conversion Ratio (and Feathering in Poultry). A continual problem in poultry production is how to get excellent growth, feed conversion ratio, meat yield, and feathering in commercial strains of birds that are continually improving due to genetic gains. Administering L-thyroxine or thyroactive iodinated casein with 1% thyroxine content (sometimes inaccurately estimated higher) has been studied. Christensen (Aug. 12, 1986; U.S. Pat. No. 4,604,968; Increasing the Efficiency of Poultry Production) disclosed that fertile poultry eggs in late stages of incubation can be treated with physiologic dosages of T₄ to augment the endogenous thyroid output of the embryos and improve hatchability rates and to have a favorable impact on mature body weight and feed conversion ratio.

Turner and Reineke (Sep. 18, 1945; U.S. Pat. No. 2,385,117; Method of Increasing the Egg Production of Fowls) stated that thyroactive iodinated casein could be added to the (presumably mash-type, unpelleted) diets of growing White Rock chickens at levels of 0.01% to 0.10% to improve growth rate and feathering. American Dairies, Inc. (Patent GB601,469, 6 May 1948) described a poultry feed formula containing thyroactive iodinated casein at a level of 0.01 to 0.10%, or equivalent on lime grit or cracked oyster shells, as a “poulty medicine”. The level of 0.01 to 0.08% was indicated for increasing the rate of growth of young fowls. Penquite et al. (1946) reported that oral administration of “thyroid” at 0.2 grain per chick per day from 0-7 days and 0.5 grain per chick daily from 7 to 49 days of age produced a very marked improvement in the color, gloss, and growth of feathers, hastening sex identification, and in the weight of the chickens. Note that 1 grain=0.065 g so the doses were 0.0130 and 0.0325 g, respectively. Glazener et al. (1949) concluded that 100 g thyroactive iodinated casein/ton of feed was optimum for growth and feed conversion ratio of New Hampshires and Barred Plymouth Rocks during growing-finishing, but higher levels (200, 300, and 400 g/ton) stimulated feathering to a greater extent. In Rhode Island Red chickens, rate of feathering increased as thyroactive iodinated casein in diet increased from 45 to 720 g/ton, then plateaued from 720 to 1,440 g/ton (Boone et al., 1950). Wilson et al. (1983) found that dietary thyroactive iodinated casein (0.01 to 0.04%) increased broiler central tail feather length at 2 weeks of age.

Majeed et al. (1984) injected male chicks of an egg-laying strain with L-thyroxine subcutaneously with 0, 1, 2, or 4 μg/100 g body daily from 7 to 12 weeks of age and found that live weight gain was increased. Suthama et al. (1989) ascertained that dietary inclusion of a level of 0.4 mg L-thyroxine/kg resulted in less abdominal fat in female broiler chickens and higher muscle protein synthesis rate in male broiler chickens. Adding a level of 1.2 mg/kg L-thyroxine to the diet improved feed conversion ratio in both sexes and produced greater muscle weight and muscle synthesis rate in male broiler chickens. Using a level of 3.6 mg/kg dietary L-thyroxine produced lower body weight gain, breast muscle weight, liver weight, and abdominal fat content in both sexes, and higher protein synthesis and breakdown in skeletal muscle of male chickens.

Dawson et al. (1996) detected in 5-month old, about half grown, farmed ostriches mean T₄ levels of 3.1 nmol/L (range 0.2 to 9.9 nmol/L), and there was a positive correlation (P<0.0005) between plasma thyroxine and body weight, which ranged from 10.8 to 51.5 kg. Plasma thyroxine was highly variable within and between individual ostriches. Blache et al. (2001) stated that thyroid function is abnormally low in ostriches and emus, and dysfunction of the thyroid axis may be the cause of their neoteny, a phenomenon in which juvenile characteristics are retained into adulthood.

Protamone® thyroactive iodinated casein (1% thyroxine content; Agri-Tech, Inc., Kansas City, Mo.) is FDA approved for improving growth rate and feathering in ducks (100 to 200 g/ton). However, marketing of this product was discontinued several years ago. The original recommended inclusion rate in broiler chicken feeds was 10 oz./ton of feed (283.5 g) according to Wheeler et al. (1948).

Marple et al. (1981) reported that metabolic body size (kg^{0.75}) of barrows and gilts, measured at 4 week intervals from 10 to 26 weeks of age, was positively correlated with thyroxine secretion rate (r=0.44). D. B. Anderson and J. F. Wagner (Apr. 4, 1989), in U.S. Pat. No. 4,818,531, Growth Hormone and Thyroid Hormone, described a method of offsetting feed intake depression in pigs, attributable to exogenous administration of a growth hormone related substance such as porcine growth hormone (10 to 300 mcg/kg/day), by implantation with or daily oral administration of a thyroxine-containing substance (e.g., 0.0005 to 0.0250% iodinated casein for 1 to 5 months). With dietary thyroprotein, the feed intake of pigs, chickens, or dairy cattle with elevated levels of growth hormone, can be increased. The administration of thyroactive iodinated casein is preferred.

Lambourne (1964) determined that fine-wool Merino wethers had increases of 34% in annual fleece weight by 60 mg thyroxine implants in summer and autumn, and a higher plane of nutrition was needed. It was concluded that the dose tolerated was dependent on plane of nutrition and that repeated implantations every season without opportunity for recovery of catabolized body tissues may produce no increase in wool growth and may cause death. Puchala et al. (2001) reported that hyperthyroidism resulting from daily injections of thyroxine at 150 mcg/kg body weight increased mohair length growth rate by 15% and decreased fiber diameter by 7.8%.

According to the present invention, growth rate and feed conversion ratio of growing animals, along with feathering in avian species, are improved by continuous feeding of appropriate low levels of thyroid hormone(s), plus magnesium (5% to 300% of dietary minimum requirement). If necessary for steam pelleting, coating of the product for thermostability to prevent denaturing and loss of potency is done by technology known in the industry.

n. Composition for Growth and Health of Integument, i.e., for Feathers, Fur, Hair, Mohair, and Wool and for Hoof Health. In special cases such as growing hackle feathers for the fly fishing industry, there is a need for a supplement suitable for improving the rate of feathering. Such a supplement according to the present invention contains: 1) iodide or iodate contributing 1 to 7 mg iodine/kg diet, and/or preferably thyroactive iodinated casein (about 100 to 720 g/ton of feed, or equivalent level of other thyroid hormone product), 2) selenium yeast (about 0.1 to 0.3 mg Se/kg of feed; 0.3 mg/kg is the legal limit), and 3) zinc methionine (about 20 to 100 mg Zn/kg of feed; preferably at least 40 mg/kg). Each of these three organic trace mineral substances is known to separately and favorably influence the rate of feathering in avian species yet this is the first invention using them together for improved results. Selenium yeast contains selenomethionine, and both of these selenium and zinc compounds provide methionine required for feathering. Various combinations of levels of the three organic trace mineral compounds are acceptable, and any two of them together make an effective composition. Optionally, a methionine source such as DL-methionine or methionine hydroxy analog (e.g., 0.025 to 0.10% of diet) for feathering and/or biotin can be added for skin and foot or hoof health.

Supplee et al. (1958) showed that zinc from ZnCl₂ was effective at correcting a feather abnormality (“thinning of the web of the secondary flight feathers”) in turkey poults. Spivey-Fox and Jacobs (1967) found that 25 mg zinc/kg diet was necessary for normal feathering in Japanese quail, 0-28 days old. Maynard and Loosli (1962) stated that the highest concentrations of zinc are in epidermal tissues such as skin, hair, and wool, and severe zinc deficiency causes parakeratosis in swine and poor feathering with keratosis in chicks. McNaughton (1991) reported lower skin lesion scores (tears and scratches) in broiler chickens fed diets with 20, 30, or 40 mg each of zinc methionine and manganese methionine per kg versus negative control or 40 mg extra zinc or manganese per kg from sulfate forms. Zinc methionine reduces foot problems (hoof lesions) in dairy cattle (Anonymous, 1990) and in breeding ewes (Anonymous, 1985). Selenium yeast has been reported to improve feathering in broiler chickens at 0.1 or 0.3 mg Se/kg diet (Edens et al., 2000; Choct et al., 2004).

Dogs with hair and skin conditions (dull coat, brittle hair, loss of hair, scaly skin, pruritis, or dermatitis) treated with approximately 5 mg biotin/10 kg body weight for 3 to 5 weeks were cured in 60% of the cases (Frigg et al., 1989). Fur-bearing animals such as mink and foxes need adequate biotin for prime pelts. High doses of biotin may have beneficial effects on skin, hair, and fingernails in humans and animals with normal biotin status. Dietary biotin has been reported to improve hoof horn condition in horses (5 mg/100 to 150 kg body weight per os daily for 8-15 months; Geyer and Schultze, 1994), white line disease lameness in dairy cows (20 mg/d in ration for 6 months; Potzsch et al., 2003), and hoof lesions in breeding sows (350 mcg/kg diet; Simmins and Brooks, 1988).

o. Decreasing Body Lipid Content and Increasing Lean Meat Yield. Hayashi et al. (Aug. 22, 1989; U.S. Pat. No. 4,858,560) revealed that both sodium iodide and sodium iodate, plus “protein with which iodine is combined”, with inclusion rate of 1 to 3,500 ppm as iodine and ratio of iodine-combined-protein at 1 to 350 mg/kg as iodine in the feed, can improve poultry meat quality to have “meager fat and much meat in the body”. Iodide to iodate preferred ratio is 1 to 10:1. As an example, a simple process was described using milk casein suspended in water to which powdered iodine was added, stirred, let stand for 24 hours, dried, and ground to produce the protein with iodine combined. Suthama et al. (1989) reported that 0.4 or 1.2 mg thyroxine/kg in feed increased broiler chicken body weight and decreased feed conversion ratio and abdominal fat, especially in females, whereas 3.6 mg/kg was detrimental. Akiba et al. (1983) noted that injections of T₄ decreased liver lipid content in chicks.

Cogburn (Sep. 14, 1990; U.S. Pat. No. 5,168,102, Endocrine Manipulation to Improve Body Composition of Poultry), improved the body composition (carcass quality) of poultry by increasing plasma levels of thyroid hormone T₃ to about 150 to 250% of normal (endogenous hormone level=100%) during essentially the finishing phase (for example, in broiler chickens about 3 to 7 weeks of age). The method lowers the extent of fat deposition and increases the proportion of protein in living poultry grown for meat production. Triiodothyronine by oral route, optimally at 0.1 to 1 ppm level in feed, was recommended. A 17-25% reduction in body fat can be obtained with finishing-phase T₃ administration alone. The fat reducing effect of dietary T₃ in the broiler chicken finishing phase occurred without substantially diminishing market weight. A level of 1,000 mg betaine/kg diet has been associated with increased breast meat yield in several experiments with broiler chickens and turkeys (Remus, 2000). Buyse et al. (2001) found that 100 mg L-carnitine/kg broiler diets reduced abdominal fat in females at 42 days of age.

Harms et al. (1982) observed that dietary iodinated casein (0.022%) with 1% thyroxine activity reduced liver fat from 32.8 to 18.6% within 28 days in caged laying hens. In a second trial, levels of 0.011 or 0.022% supplement reduced liver fat from 26.5 (control) to 22.1 to 15.0% on normal diet and from 29.0 (control) to 17.3 to 12.2% on higher (+17% nutrient density) diet within 56 days.

ZiRong et al. (1999) found that pigs supplemented with 1,000 mg betaine/kg diet grew fastest with 13.20% increase in average daily gain and 7.93% decrease in feed conversion ratio. Betaine elevated serum T₄ and T₃. Owen et al. (1994) observed that 50 mg L-carnitine/kg of diet increased longissimus muscle area and slightly reduced backfat thickness and daily lipid accretion rates in growing-finishing pigs. Zabaras-Krick (1997) reported lower feed conversion ratio and backfat thickness and greater loin eye muscle area in pigs fed diets with 1-1.25 kg betaine (97% purity)/tonne.

Protamone® thyroactive iodinated casein (1% thyroxine content; Agri-Tech, Inc., Kansas City, Mo.) is FDA approved for hypothyroid (often obese) dogs in the form of 1 g tablets, containing 25 mg of thyroactive iodinated casein, and dosed once per treatment using 1 tablet per 10 lb of body weight. However, marketing of this product was discontinued several years ago.

According to the present invention, thyroid hormones are administered in combination with magnesium (5% to 300% of minimum requirement), and optionally, betaine (about 800 to 1,000 mg/kg diet) and/or L-carnitine (30 to 50 mg/kg diet) to reduce body lipid and increase lean content.

p. Increasing Iodine Content of Meat, Eggs, and Milk with Exogenous Thyroid Hormones. Associated with supplementation of animal diets with exogenous thyroid hormones is the possibility that tissues such as muscle and liver will retain some of the iodine, perhaps allowing an opportunity for marketing of iodine enriched meat for humans or rendered byproducts for animals. He et al. (2002) fed 17 kg pigs diets with potassium iodide which contributed 5 or 8 mg iodide/kg feed for 3 months and found that the iodine content increased in fresh muscle by 45%, in adipose tissue 213%, in heart by 124%, in liver by 207%, and in kidneys by 127%. In the iodide supplemented groups, there was a significantly higher concentration of thyroxine (T₄) and a lower concentration of triiodothyronine (T₃) in serum.

Iodine is accumulated in the egg yolk during oogenesis (yolk formation). Iodine-enriched eggs produced during periods of supraoptimal levels of iodine supplementation are marketable as “designer eggs” (e.g., Eggland's Best eggs today). Dried seaweed (kelp) is an AAFCO (2003) feed ingredient containing iodine. Kaufmann et al. (1998) investigated feeding diets containing seaweed, contributing 2.5 or 4.9 mg iodine/kg of complete feed, and found that iodine concentration in eggs increased significantly with extra iodine intake after a 2-week period compared to unsupplemented control feeds. A human egg consumption study revealed that eggs enriched with iodine can increase iodine excretion and therefore improve iodine supply and status in man. Rys et al. (1997) compared iodine deposition in eggs of chicken hens and partridge hens fed iodine from kelp (seaweed; 0, 2.0, or 4.4 mg iodine/kg feed) or calcium iodide (0, 2.7, or 7.2 mg iodine/kg feed). Iodine from kelp passed into eggs more effectively than iodine from calcium iodide. Christensen (1985) injected L-thyroxine (50 ng, a physiological dose) into fertile turkey hatching eggs at 25 days of incubation and observed significantly improved hatchability. He concluded that hypothyroidism may be a cause of poor hatchability among turkey eggs.

During lactation in mammals, injected or dietary thyroid hormones alone or in combination with other sources of iodine increase the iodine content of milk. For example, Grace and Waghorn (2005) injected dairy cows intramuscularly 3 times with iodized oil (2,370 mg iodine/dose) at the beginning of lactation and about 100 days apart. Milk iodine levels increased from <20 mcg/L (0.24 mg iodine/kg pasture dry matter) to 160 and 211 mcg/L at least 55 days after each treatment.

According to the present invention, thyroid hormones T₃ (about 0.1 to 2.5 mg/kg diet), T₄ (0.5 to 10 mg/kg diet), or both (proportional levels), are administered to enhancing tissue iodine content (e.g., thyroxine contains 65.34% iodine in an organic form) as well as providing other benefits. Exogenous thyroid hormone(s) are administered to adult females of avian species, particularly chicken hens, as sources of organic iodine (e.g., thyroxine has 65.34% iodine) to produce iodine-enriched table or fertile eggs. Milk iodine content is increased by dosing dairy cattle, dairy goats, breeding ewes, sows, and other mammals with thyroid hormones by diet or injections. In each case, optionally the thyroxine supplement is given in combination with iodide or iodate compounds.

q. Improve Semen Quality and Sperm Characteristics. The turkey and dairy industries primarily utilize artificial insemination instead of natural mating, and one problem is finding better diluents and extenders to mix with fresh or frozen semen. Schultze and Davis (1948) showed that the addition of DL-thyroxine to bull semen increased the oxygen (O₂) consumption by the spermatozoa, and Schultze and Davis (1949) demonstrated that the addition of L-thyroxine to bull semen improved conception rate of cows as measured by a decrease in early embryonic mortality. Maqsood (1954) observed that the addition of DL-thyroxine to bull semen increased oxygen consumption as well. Carter (1932a) found that thyroxine stimulates the action of secretion from ripe ova of two species of Echinus in that the spermatozoa were activated and had their life prolonged when placed in a 1:50,000 solution of thyroxine and seawater. Carter (1932b) reported that the addition of thyroxine to rabbit semen improved the oxygen consumption rate of the spermatazoa. Eiler and Armstrong-Backus (1987) injected bulls with various levels of T₄ or T₃ and found that seminal concentrations of the hormone increased within 120 minutes. It was concluded that exogenous thyroid hormones passed from blood to the ejaculate, with T₃ passing faster than T₄. According to the present invention, a thyroid hormone(s) is added to semen to increase oxygen in combination with substances to improve sperm characteristics.

r. Increasing Milk Yield in Dairy Cattle, Dairy Goats, Sows, and Other Lactating Animals. Vetoquinol Canada in Quebec holds rights to a Canadian government approved but no longer marketed vitamin and trace mineral premix (Extralac) containing thyroactive iodinated casein for lactating sows. It provides 227 mg thyroactive iodinated casein/kg complete feed for lactating sows at 28.35 g premix per head per day, 3 days before farrowing through to weaning. No magnesium is included in the premix.

Protamone® thyroactive iodinated casein (1% thyroxine content; Agri-Tech, Inc., Kansas City, Mo.) is FDA approved for improving milk yield in dairy cattle (0.5 to 1.5 g per ton feed per 100 pounds body weight). It is for the declining plane of lactation, must be accompanied by increased feed intake, and may increase sensitivity to heat (i.e., thermal stress). However, marketing of this product was discontinued several years ago. Shaw et al. (1975) found that dairy cows fed 15 g thyroprotein/head/day for 5 or 13 weeks had increased serum thyroxine from baseline of 54 ng/mL to a peak of 135 ng/ml at 6 days after thyroprotein feeding, then serum T₄ declined to about 80 ng/mL at 23 days. For 5 weeks of treatment cows averaged 2.2 to 3.3 kg/day and for 13 weeks of treatment cows averaged 0.95 to 2.5 kg/day more milk than controls.

Dietary L-carnitine at 50 mg/kg for breeding sows during advanced stages of gestation allows accumulation of more body fat reserves, higher piglet birth weights, more weight uniformity within litters, and lower piglet mortality. At 30-50 mg/kg in lactating sow diets, L-carnitine reduced weight loss and shortened the interval between weaning and first return to service (Baumgartner and Alonso, 1999). Administering 50 mg L-carnitine per boar daily yielded one more sow insemination per ejaculation. Baumgartner and Blum (1998) found that 30-50 mg/kg diet was best for pigs weaned at 28 days, but higher level (50 mg/kg) may be needed for earlier weaned pigs. They recommended 100-200 mg/kg diet for boars, 50 mg/kg for sows in gestation and lactation, and 500 mg/kg for piglet milk replacers.

According to the present invention, L-thyroxine or a thyroxine-containing substance, in combination with magnesium (5% to 300% of requirement) to support increased metabolic rate (enzyme activity) and help keep blood Mg levels normal, and optionally with L-carnitine (30-50 mg/kg diet) especially for sows, is administered to increase milk yield of mammals.

s. Disease Challenges and Metabolic Disturbances Causing Decreased Blood Thyroxine. Rudas et al. (1986) discovered that when day-old broiler chickens were infected with intestinal homogenates from chickens suffering from malabsorption (“runting and stunting”) syndrome, serum thyroxine was lower from days 6 to 29 and body weight was lower within one week after inoculation compared to controls. Thyroid function is one of the earliest targets of this syndrome. Scheele et al. (1992) observed in both a normal broiler strain and one selected for fast growth and low feed conversion ratio, but more sensitive to heart failure and ascites, that high-fat diets (i.e., polyunsaturated fatty acids) inhibited the extra thyroidal (e.g., peripheral tissues) conversion of T₄ to T₃ decreasing heat production and retained fat energy. Limited thyroid hormone production and a lower capacity for oxygen consumption could be two of the factors initiating hypertensive pulmonary syndrome and ascites in broiler chickens.

Dewil et al. (1996) determined the plasma T₄ concentration in late incubation chick embryos of an ascites-susceptible broiler strain to be lower than plasma T₄ of an ascites-resistant broiler strain. Gonzales et al. (1999) stated that changes in plasma thyroid hormone concentration in direct response to selection for low feed conversion ratio and fast growth may be causatively linked to susceptibility for metabolic disturbances such as sudden death syndrome and ascites. Luger et al. (2001) observed that in ascitic broilers exposed to low ambient temperature and pelleted feed (high mortality rates of 24.3 and 24.2% in two trials to 49 days of age), plasma T₄ concentration declined significantly during the week of death but not in all cases. Luger et al. (2002) noted that ascites (21.5%) induced in fast-growing broiler chickens by relatively low ambient temperature and pelleted feed was significantly reduced (to 7%) by exogenous L-thyroxine. Malan et al. (2003) reported, based on an experiment with 7 genetic lines (2 pure sire, 2 pure dam, 2 slow-growing, and commercial broiler lines), that fast-growing breeder sires had lower plasma thyroid hormone, proportional lung weights, and arterial pO₂, and higher arterial pCO₂ pressures than the slow-growing lines. Ascites incidence was associated with lower heat production and oxygen requirement per unit of metabolic size. Buyse et al. (2001) found that 100 mg L-carnitine/kg in broiler diets reduced abdominal fat in females at 42 days, increased circulating T₃ levels, and greatly increased absolute and proportional heart weight without right ventricle enlargement, making the additive potentially useful for ascites prevention.

According to the present invention, the problem of low thyroid hormone levels in avian species afflicted with various disease or metabolic conditions is ameliorated or completely corrected, by dosing with exogenous thyroid hormones. Relatively low levels of administered thyroid hormones may bring circulating levels up to normal. Optionally, L-carnitine is co-administered with them.

t. Brooding and Cool Stress (Increased Basal Metabolic Rate and Heat Production). Poultry hatchlings brooded under lower than optimal temperatures (e.g., 88° F. versus 94° F. to save on fuel expense commercially) are more susceptible to ascites and to increased mortality and morbidity (e.g., respiratory diseases). According to the present invention, exogenous thyroid hormones are administered to stimulate metabolic rate and body heat production in those special circumstances when conditions are not optimal.

Stahl et al. (1961) stated that even though cold stress activates the pituitary and thyroid gland within a matter of hours in guinea pigs and rabbits, 5-month old New Hampshire pullets exposed to 4.4° C. (40° F.) had no significant change in thyroidal-I¹³¹ release rate in 8 hours, thus putting the chickens in jeopardy. Poczopko and Uliasz (1975) discovered in fasting male goslings of 3, 10, and 21 days of age, exposed to 6 hours of cold (5° C. or 41° F.), that a single subcutaneous injection of L-thyroxine (100 mcg/kg body weight) maintained normal metabolic rate during 4 hours of measurement whereas fasted untreated control goslings showed a 17% decrease in metabolic rate during the same time. Goslings at 5 to 7 days of age did not respond to 100 mcg/kg body weight injections of thyroxine for 4 days whereas at 22 to 24 days of age metabolic rate was increased. Jastrzebski and Barowicz (1975) observed that cold (11° vs 28° C.) increased thyroid weight by 7% in 8-week old Cornish chickens. Kuhn et al. (1984) revealed that in posthatch chicks more T₃ will be generated in cold-exposed birds and more reverse T₃ will be produced at higher ambient temperatures.

u. Ameliorating Effects of Heat Stress. The levels of circulating thyroid hormones are decreased with increasing ambient temperatures so that the environment provides more of the body heat and less is derived from metabolism. This deficit in thyroid hormones under heat stress may negatively impact performance due to the involvement of thyroid hormones in many metabolic processes. Although it may be counterintuitive, according to the present invention thyroid hormone is administered at low levels to poultry or other animals in heat stress to improve live performance, and optionally this is done in combination with magnesium, and L-carnitine, sodium salicylate, or both. Acclimation to heat stress is a known phenomenon. Administering thyroxinic substance(s) during the first week of life to young poultry, causing excess body heat for about 12 to 24 hours or more than one event, as if internally rather than externally (i.e., with high ambient temperatures) exposing the birds to higher body temperatures temporarily, may improve heat stress resistance at later ages (e.g., better growth and less mortality).

Summer sterility of rams may be improved by thyroxine or synthetic thyroprotein administration (Berliner and Warbritton, 1937; Bogart and Mayer, 1946). Kamar (1960) reported that thyroactive iodinated casein (˜1% thyroxine) supplemented to White Leghorn and White Baladi cockerels at levels of 0.011% or 0.017% improved general semen characteristics including volume, concentration, and numbers per ejaculate over the controls whereas the 0.006% and 0.022% levels were less effective during summer heat stress in Egypt. L-carnitine supplemented orally to pigeons at 90 mg/bird daily reduced the increase in heat production during electrostimulation by improving fatty acid oxidation efficiency during heavy exercise (Janssens et al., 1998). Aspirin added at 500 or 1,000 mg/kg laying hen diet during heat stress deceased serum T₄ and T₃ levels but increased egg production, feed conversion ratio, and egg shell weight and thickness (XiaoTing and YouMing, 2002).

v. Increasing and Sustaining Egg Production and Improving Egg Shell Quality. Turner and Reineke (Sep. 18, 1945), in U.S. Pat. No. 2,385,117, Method of Increasing the Egg Production of Fowls, fed thyroactive iodinated protein to normal fowls such as chickens, turkeys, ducks, and geese at levels of 0.01 to 0.04% of the diet for increasing and sustaining egg production. It was stated that stimulation of egg production with the supplement occurs during seasons of normally low or reduced production and with advancing age. A level of about 0.022% (200 g/ton) supplement was added to the diets of adult fowls of both sexes to favorably influence male reproduction (sperm) or egg production. The supplement was designed for use in mash feeds not steam pelleted. American Dairies, Inc. (Patent GB601469, 6 May 1948) described a chicken feed formula containing thyroactive iodinated casein at a level of 0.01 to 0.10%, or equivalent levels on lime grit or cracked oyster shells, as a “poulty medicine” for increasing the egg production of fowls. The level of 0.01 to 0.04% was indicated for increasing and sustaining egg production. Gutteridge and Novikoff (1947) fed breeder hens diets supplemented with 0 or 200 g thyroactive iodinated casein/ton for 6 months and observed an increase in egg specific gravity (shell quality) with the additive. Wheeler et al. (1948) indicated that approximately 200 g thyroactive iodinated casein/ton is satisfactory for laying hens.

Maruta and Miyazaki (U.S. Pat. No. 6,660,294, Dec. 9, 2003, Poultry Eggshell Strengthening Composition) demonstrated 5.2% increases in egg shell thickness of caged laying hens fed Bacillus subtilis C-3102 spores at about 0.003% of diet. Lactic acid produced by the Lactobacilli which proliferate due to Calsporin supplementation can also be added directly to the diet of laying fowls (e.g., 0.5% of diet). Rabie et al. (1997) fed 50 to 500 mg L-carnitine/kg diet to 65-week-old laying hens for 8 weeks and got improved albumen quality (i.e., height and Haugh units) whereas egg white % increased and egg yolk % decreased.

According to the present invention, L-thyroxine or thyroxine-containing substance is administered additionally with magnesium, 25-hydroxy-vitamin D₃ (20-69 mcg/kg diet), Calsporin (spores at 0.003%), and/or L-carnitine (50-500 mg/kg diet), and if necessary, with coating of the product for thermostability through steam pelleting, representing a significant improvement suitable for modern poultry strains and feed manufacturing conditions.

w. Increasing Bone Breaking Strength in Caged Laying Hens. Fragile bones in spent laying hens which shatter and splinter on handling is a well known problem at chicken processing plants. Rowland and Harms (1970) demonstrated that a level of 0.062% iodinated casein (1% thyroxine activity) in laying hen feeds (3% calcium) for one week increased bone breaking strength in 66-week old White Leghorn males (43.99 vs 40.84 kg force) and females (25.37 vs 23.27 kg force) compared to unsupplemented controls. In a series of two trials using 0, 0.062, 0.124, 0.187, 0.249% levels of iodinated casein, male and female combined average bone breaking strength values were 14.88, 16.16, 16.39, 16.75, and 17.14 kg force. The 0.187% supplement level with +3% (6% total) calcium had 17.86 kg bone breaking strength (left tibia). According to the present invention, a dietary thyroid hormone, such as in thyroactive iodinated casein (e.g., 0.050-0.075%), in combination with magnesium supplement (5% to 300% of minimum requirement), and/or 25-hydroxy-vitamin D₃ (35-69 mcg/kg diet), is administered to adult aged male or female poultry, especially those housed in cages, to increase bone strength prior to catching, livehaul, and processing.

x. Body Weight Control, Maintenance, or Restriction. According to the present invention, a moderate level dietary thyroactive substance, such as 0.2 to 10 mg L-thyroxine/kg of feed, which stimulates metabolism and depresses appetite, optionally in combination with magnesium, is administered to control, maintain, or restrict body weight of growing broiler or turkey breeder replacements. These classes of poultry are genetically designed for heavy muscling and fast growth, but for breeding purposes need to have body weight curtailed during growth and as adults by more or less continual feed restriction. Cherry and Savage (1974) reported linear decreases in body weight of broiler-type chicks at 3 or 4 weeks of age when diets were supplemented with 0, 0.05, 0.10, 0.20, and 0.30% thyroactive iodinated casein. Newcomer (1976) demonstrated that body weight of young male White Leghorn chicks from 2 weeks to approximately 3, 4, or 7 weeks of age was depressed with 0.02 or 0.04% dietary iodinated casein compared to control. Significant differences in body weight were observed within 2 weeks. Harms et al. (1982) found that 0.011 or 0.022% thyroactive iodinated casein added to the diet of caged laying hens significantly reduced body weight at 28 days (1460 vs 1346 g at 0.022% in trial 1) or 56 days (1576 vs 1460 or 1461 g and 1593 vs 1472 and 1447 g for 0.011 and 0.022% respectively in trial 2). Coincident with this were reductions in liver weight and fat content. Leung et al. (1985) observed that growing cockerel and pullet chickens fed 10 mg/kg dietary thyroid hormone had a 55.24% reduction in body weight gain with triiodothyronine (T₃) or a 28.18% reduction with L-thyroxine (T₄) compared to controls. The T₃ was more active than T₄ in reducing growth and was toxic when feed at 10 mg/kg both in cockerels and pullets.

y. Improving Reproductive Performance of Males: Weight Control and Molting Process. Crew (1925) was successful in rejuvenating 5 roosters 5 to 8 years old by administering desiccated thyroid (containing both T₃ and T₄) by mouth each day over a 6-month period. The dosage was equivalent to 0.2 mg iodine each day for the first two weeks, 0.4 mg iodine daily for weeks 2-4, and 0.8 mg iodine per day for the rest of the 6-month experimental period. Crew did not report the actual fertility records of these males, but they were able to fertilize the eggs of hens in natural matings. Following the administration of thyroid, the birds without exception promptly molted. Hill (1935) observed that the hypophysectomized (i.e., pituitary removed) rooster began to molt 2 to 4 weeks after the operation and remained in a perpetual state of molt. Titus and Burrows (1940) fed 6-month old White Leghorn cockerels 100 mg desiccated thyroid 3 times a week of 5 weeks. The excessive thyroid feeding caused semen production (measured three times weekly) to decrease steadily and at a fairly rapid rate, and the decrease continued for several days after the feeding of thyroid was discontinued.

Jaap (1933) fed adult mallard drakes desiccated thyroid through the winter and early spring months. The daily dosage ranged from 0.25 to 1.00 g of desiccated thyroid per duck. Testis size increased from 2 to 10 times that of the controls. There was a marked increase in spermatogenesis. Jaap explained the results on the basis that thyroid greatly increased the metabolic processes which resulted in a greater elimination of testis hormone from the body (and testes responded by enlarging to meet demand for testosterone). Turner and Reineke (Sep. 18, 1945), in U.S. Pat. No. 2,385,117, stated that a level of about 0.022% (200 g/ton) thyroprotein added to the diets of adult male fowls favorably influenced male reproduction (viable sperm output) or to adult females increased egg production. Himeno and Tanabe (1957) injected two cockerels i.m. with 4 mg of L-thyroxine every 2 days until a total amount of 20 mg was reached. As a result, cockerels started to molt severely in primary feathers and body feathers.

Lien and Siopes (1991) reported from a lighting study (14 vs 8 hours of light per day) to molt normal or thyroidectomized turkey breeder males that the molt and termination of semen prod-uction occurring in response to the shorter day length were inhibited by thyroidectomy. This indicated that thyroid hormones are involved in the molting process in male turkeys. It has been shown from commercial experience with Hy-Line W-36 White Leghorn breeding stock that males can be put through a molting process (i.e., short days and feed restriction) along with hens to improve subsequent fertility of eggs produced by the flock. The males have body weight loss but little or no observable feather loss whereas females lose body weight, shed feathers, and cease egg production (Javad Farahani, Iran, personal communication Sep. 9, 2004, ja_farahani@yahoo.com).

Hays (1948) administered thyroxine to high-fecundity Rhode Island males of various ages (12 to 48 months). Thyroxine (1 mg) tablets were given to each male by mouth 3 times weekly from January 8 to March 14 (29 mg total). No significant effects on fertility (80 to 100% by periods) were found due to either factor in young or old males in natural matings. Craige (1954) thought that low thyroxine output disrupted the reproductive organs, and in some instances spermatozoa may be shed into the lumen and ejaculated before they have matured. Maqsood (1951a, b) found that administration of thyroxine to infertile rabbit bucks increased the fertility of these males and reduced the incidence of a “peculiar type of sperm abnormality”. Jiang et al. (2000) studied adult rdw infertile male rats and found that thyroxine treatment markedly increased circulating serum T₄ levels and the weights of both epididymides and testes, and decreased the percentage of epididymal sperm with cytoplasmic droplets compared to untreated rats. Infertility of epididymal sperm was completely reversed by exogenous T₄ when determined both in vivo and in vitro, and homozygous embryos developed to term after transfer without loss of viability. The European Commission (2002) stated that many environmental agents interefere with thyroid function, the most prominent effect being the development of goiter, but decreases in T₃ and T₄ may also alter brain maturation and testis development.

Jacquet et al. (1993) fed 96-week old broiler breeder cockerels diets with 0, 2, or 5 ppm T₄ for 4 weeks and found that plasma testosterone levels and daily sperm output returned to control values at weeks 5 and 11, respectively. Broiler breeder roosters tend to get so heavy in weight by around 40 weeks of age that their libido and mating activity decline, and correlated with this, the fertility of hatching eggs decreases. The commercial broiler breeder industry usually “spikes” the flocks with extra younger males to continue to get acceptable fertility through the first cycle of hatching egg production lasting to about 65 weeks of age.

According to the present invention, adult males of avian species are either fed continuously a low level of thyroid hormones (e.g., 0.2 to 5 mg L-thyroxine/kg diet) or induced into a reproductive quiescence (male molt period) preferably by use of exogenous T₃ and T₄ (about 5 to 20 mg/kg diet), optionally in combination with magnesium, exposure to preconditioning short day length, short day length during molt induction, and low nutrient density diet. Exogenous thyroid hormones are administered in low to moderate doses (e.g., about 0.5 to 10 mg L-thyroxine/kg diet) prior to sexual maturity in avian males or females to delay sexual maturity (egg or semen production) when such postponement is beneficial.

z. Transient Hypothyroidism Subsequently Boosts Testes Size and Semen Production in Males. A problem in commercial poultry production is loss of fertility in aging male breeders. Surprisingly, inducing hypothyroidism (e.g., with an iodine deficient diet containing a natural goitrogen) at a critical age in testicular development, according to the present invention, results in subsequent improvements in spermatogenesis and persistency of fertility in poultry and other animals.

Cooke and Meisami (1991) treated male rats from birth to day 25 with 6-propyl-2-thiouracil and noted that body weight decreased but testis weight increased by 40%, 60%, and 80% at 90, 135, and 160 days, respectively, compared to controls. Lesser increases were found in weight and DNA content of epididymis and accessory organs. Testosterone levels were unchanged. Neonatal hypothyroidism in rats resulted in lasting enlargements in the ultimate size of testis and other reproductive organs in the adult. Cooke et al. (1991) obtained testes and epididymis from rats in the foregoing study and found sperm motility and concentration in the caudal epididymal fluid of adult males previously treated with thiouracil were normal, and males were fertile and sired litters having normal pup numbers and weights. Neonatal hypothyroidism was associated not only with increased testis size but also with increased efficiency of sperm production. Maximum sperm production was reached at 160 days of age in treated rats compared to 100 days in controls, coinciding with the attainment of final testicular size. Results of these papers, as well as Cook et al. (1992), indicate that transient neonatal hypothyroidism markedly increases both testicular size and sperm production in the adult rat without loss of sexual behavior. Cooke et al. (1994) stated that in male rats transient neonatal hyperthyroidism decreases Sertoli cell proliferation and ultimate testis size whereas transient neonatal hypothyroidism causes prolonged Sertoli cell proliferation, delayed Sertoli cell maturation, and increased adult Sertoli cell number, testis weight, and sperm production. Joyce et al. (1993) observed that epididymal sperm from transient hypothyroid mice were motile and morphologically normal at 90 days.

Kirby et al. (1992) fed thiouracil to neonatal male rats from birth to 24 days of age and found a 68% increase in testis size at 100 days. Serum testosterone levels were unaffected, but circulating levels of FSH from the anterior pituitary were chronically reduced. Serum T₄ and T₃ levels returned to control levels within 15 days after removal of thiouracil. Administering 6-propyl-2-thiouracil to suckling rat pups from birth to 24 days postpartum as a 0.1% solution in the mother's drinking water, Hardy et al. (1993) observed that the number of Leydig cells per testis at 180 days increased by 69% in thiouracil-treated compared to control rats, whereas the average Leydig cell volume declined by about 20%. LH-stimulated testosterone production was reduced by 55% in Leydig cells from treated rats, commensurate with a 50% decline in the number of hCG-binding sites (i.e., LH receptors) in these cells. These results clearly showed that the dramatic increase in adult Leydig cell number after neonatal thiouracil treatment was counterbalanced by a permanent decline in Leydig cell steroidogenic function, producing no net change in peripheral testosterone levels

Kumaran and Turner (1949) fed several levels of thiouracil to cockerels from 1 day to 16 weeks of age to induce hypothyroidism. Thiouracil feeding depressed the testis weight slightly up to 8 weeks of age. This effect was then reversed so that by the 14th week the weights of the testes of the thiouracil group exceeded controls by about 10 times. This was accompanied by some disorganization of spermatogenic elements. Spermatogenesis was not delayed in 12-week old cockerels fed 0.3% thiouracil for 4 weeks even though testis and comb weights were reduced. Kirby et al. (1996) fed male Peterson broiler breeder chicks 0.1% dietary 6-N-propyl-2-thiouracil for 6 weeks that began at 2 week intervals (2-8, 4-10, 6-12, 8-14, and 10-16 weeks of age) and after photostimulation at 20 weeks took testis samples at 28 weeks. Roosters fed thiouracil from 6 to 12 weeks of age had a 96% increase in mean testis weight at 28 weeks (39.3 g for thiouracil group vs 20.0 g for control group) with normal morphology and increased relative sperm pro-uction. Treating birds at 8 to 14 or at 10 to 16 weeks of age increased testis weight by about 35% (27.7 g or 27.7 g versus 20.0 g) and caused precocious puberty and abnormal spermatogenesis.

Fallah-Rad et al. (2001) used 6-propyl-2-thiouracil at 15 mg/kg body weight daily from 6 to 12 weeks of age in Suffolk ram lambs to induce transient hypothyroidism. Testes were examined at 36 weeks of age, the time of castration. Testosterone levels were unaffected. Scrotal circumference was greater in treated lambs from week 26 to week 36. Treated lambs produced viable spermatozoa earlier than did control lambs (i.e., puberty was advanced in treated lambs). At week 36, sperm concentration in treated lambs was higher than in controls, but semen volumes were similar. The diameter of seminiferous tubules in treated lambs was larger than in controls. Klobucar et al. (2003) treated drinking water of male pigs (boars) with 0.1% 4-propyl-2-thiouracil from 3 to 6 weeks of age, and treated pigs were hypothyroid by 6 weeks of age. Boars were castrated at 8 to 20 weeks of age. Testis weight was slightly but significantly reduced from 8 to 12 weeks without any change in volume, and by 20 weeks testis weight was normal. Apparently the species specific critical period of testicular development in male pigs was missed in this study.

Young male Nile tilapia fish (Oreochromis niloticus), approximately 1 g weight and 3.5 cm total in length, were treated for 40 days with 100 or 150 mg thiouracil/kg diet. By 98 days treated and control tilapia had similar body weights and total lengths, but testis weight, gonad-osomatic index (testis mass/body weight), seminiferous tubules area, number of Sertoli cells and germ cells per cyst, and number of Leydig cells per testis were approximately 100% higher in treated tilapia. Nuclear volume and individual Leydig cell volume were lower in treated tilapia (Matta et al., 2002).

Neither thiouracil nor methimazole is approved for use in food-producing animals so the present invention provides an alternate approach involving administration of diets deficient in iodine, magnesium, and selenium and, if necessary, offered in combination with goitrogens, purified or in natural feedstuffs, to induce transient hypothyroidism during the species-appropriate critical period of testicular development to enhance subsequent reproductive performance in males.

aa. Protecting Animals against Overexposure to Radioactive Iodide. Poultry flocks, other animals, and human caretakers exposed to nonlethal but threatening levels of certain types of nuclear material that contain radioactive iodide (primarily ¹²⁴I through ¹³¹I) can be protected from thyroid abnormalities by daily oral doses of iodide to provide an inorganic iodide level in serum of 10 micrograms/100 cc (based on human research; Blum and Eisenbud, 1967). Optimum effectiveness requires ingestion before exposure to radiation. In humans, daily doses of 100 to 200 mg iodide (from potassium iodide—KI) per person were found to be over 97% protective (Blum and Eisenbud, 1967), and 30 mg iodide daily was only slightly less effective than 100 mg (Becker et al., 1984). The protective effect of consumed iodide is transitory and diminishes over 24 to 48 hours so daily doses are recommended. Unless exposure continues, treatment for not more than 7 to 14 days is contemplated (Becker et al., 1984). The U.S. Department of Health and Human Services (2001) recommended these daily intakes of KI to counteract radioactive iodine in humans: over 40 years, unless large exposure (>500 cGy) no supplement; 18 through 40 years, 130 mg; 3 through 18, 65 mg; children over 1 month through 3 years, 32 mg; and birth through 1 month, 16 mg.

Therefore, birds and animals which have thyroid glands should receive proportional doses of KI based on metabolic body size (kg^{0.75}). Birds and other animals can be protected from thyroid damage by bolstering blood serum inorganic iodide level to approximately the same level as that which is protective for humans. Hoshino et al. (1968) revealed that dietary iodocasein (0 vs 0.05%), manufactured in Japan, reduced thyroid uptake of ¹³¹I from 3.22 to 0.36% of dose (88,636 to 9,999 counts per minute) in Nicholas commercial chickens at 7 weeks of age. When chickens received iodocasein, serum inorganic and protein-bound radioiodine were lower by 44.9 and 92.0% respectively, the production of iodothyronines was inhibited, and thyroids showed hypofunction.

Dugrillon and Gartner (1992) observed that treatment of isolated porcine thyroid follicles with docosahexaenoic acid (DHA, C22:6, n3) at 100 and 300 microM concentrations significantly enhanced the inhibitory effect of 10 microM of iodide on thyroid follicle proliferation (45±4% versus 84±2%). Baker et al. (2003) indicated that supplemental iodide levels of 1,000 to 1,500 mg/kg cause severe growth depressions in young chicks that could be totally reversed by dietary addition of 50 or 100 mg/kg bromine from NaBr. The authors concluded that nuclear accidents or terrorists actions that result in thyroid cancer or goiter may benefit from the use of NaBr as a therapeutic agent.

According to the present invention, improvements to the well known human use of potassium iodide for overexposure to radioactive iodine for protecting animals include: 1) temporarily supplementing feed and/or water with potassium iodide, or other inorganic iodide source or EDDI, for uptake by thyroid tissue and blockade or dilution of the radioiodide effect, 2) in combination with docosahexaenoic acid (DHA, C22:6, n3) to provide 100 to 300 microM concentrations in blood to enhance the effect of iodide; or 3) instead of potassium iodide, administering sodium bromide (NaBr) at not more than 50 to 100 mg/kg of diet, or alternately reduced levels of both potassium iodide and sodium bromide, and 4) optionally, along with either potassium iodide plus DHA and/or sodium bromide plus DHA, administering exogenous thyroid hormones preferably from a product such as desiccated thyroid powder (or rendered thyroid tissue product) or thyroactive iodinated casein containing both T₃ and T₄ at daily requirement (i.e., thyroid secretion rate) to help maintain normal metabolic functions during the stress.

Accordingly, a composition or method according to the invention can be seen in any of several embodiments, as evidenced in the following examples.

### EXAMPLE 1

Use: To induce molting in wild pest bird populations by administering exogenous thyroid hormones (preferably, T3 and T4 combined for males and females or T4 alone for females only), or repeated administration as necessary, by elevating blood thyroxine levels, to limit production of fertile eggs as a nonlethal method of controlling populations; optionally in combination with Nicarbazin, conjugated linoleic acid (CLA), or both; optionally, add L-thyroxine to water for long-term (e.g., 1-5 mg/L) or short-term (e.g., 3-15 mg/L) for immediate effects.
Animals: Adult wild birds of species that are pests, especially seagulls, pigeons, blackbirds, grackles, starlings, crows, sparrows, and waterfowl (ducks, geese, etc.).
Sex: Both sexes.
Age or Stage of Production: Preferably during egg-laying season or seasons when nesting and mating.
Dose or Inclusion Rate: Mixed populations of adult males and females, preferably TIC containing T3 and T4 to provide 40 mg T4/kg diet, or adult females preferably T4 at 40 mg/kg diet from L-thyroxine or thyroxine containing substances (males have a low tolerance, <25 mg/kg diet, for thyroxine (T4) alone which in excess causes mortality; in drinking water long term (e.g., 1-5 mg T4/L) or immediate short-term (e.g., 3-15 mg T4/L) responses.
Administration: In feeds, supplements, or bait; in drinking water.
Usefulness: Nonlethal method of reducing wild pest bird population involves administering exogenous thyroid hormones in feeds, supplements, or baits, or via the water, optionally in combination with Nicarbazin and/or conjugated linoleic acid(s). The effects are to reduce the production of fertile eggs by hyperthyroidism, to impair male reproduction (decrease sperm output), and lower the hatchability of any eggs that do get laid and incubated.

### EXAMPLE 2

Use: To provide dietary iodine to animals in an organic iodine form as exogenous thyroid hormones to prevent iodine deficiency symptoms; optionally, in combination with inorganic iodide or iodate, or EDDI.
Animals: All animals including birds.
Sex: Both sexes.
Age or Stage of Production: All ages, stages of life, and phases of production.
Dose or Inclusion Rate: 0.1 to 5.0 mg/kg feed.
Administration: Preferably in trace mineral premix or vitamin premix; can be used with iodine, iodide, and/or iodate.
Usefulness: Prevents iodine deficiency; greater toxicity than inorganic iodine compounds; thyroxine by weight has 65.34% iodine; would need FDA and AAFCO approvals; there are organic forms of other trace minerals such as Zn, Mn, Fe, Cu, and Se but not iodine.

### EXAMPLE 3

Use: To ameliorate or overcome effects of antithyroid or goitrogenic substances in the diet, which diminish blood thyroid hormone and/or iodine levels, by administration of exogenous thyroid hormones.
Animals: All animals including birds.
Sex: Both sexes.
Age or Stage of Production: All ages, stages of life, and phases of production.
Dose or Inclusion Rate: Depends on depression in blood thyroid hormone levels; generally, 0.5 to 10 mg L-thyroxine per kg diet as levels above this range may be excessive; normally about 1:4.22 ratio of T3:T4 as in porcine thyroid as a general rule for estimating dose levels, and T3 or T3 and T4 can be administered.
Administration: Preferably added to the diet and optionally in combination with iodolactones or iodoaldehydes to help minimize thyroid enlargement (goiter); delta-iodolactone at 0.05 to 0.5 microM in vitro; also docosahexaenoic acid (DHA) at 100 to 300 microM in vitro; specific compounds: 6-delta-iodolactone, 14-omega-iodolactone, alpha-iodohexadecanal, 2-iodohexadecanal.
Usefulness: Helps keep blood thyroid hormones within normal ranges in cases when animals are ingesting substances that lower circulating levels and negatively impact performance; a caution is that if the intake of antithyroid or goitrogenic substances are variable, it is difficult to precisely set the counteracting dietary thyroid hormone levels.

### EXAMPLE 4

Use: To improve growth rate and feed conversion ratio in young growing animals, feathering in avian species, and wool growth in sheep or mohair growth in mohair goats
Animals: All young (non-adult), growing animals, monogastric or ruminant for growth and feed conversion; all avian species for feathering; sheep in wool production; mohair goats in mohair production.
Sex: Both sexes.
Age or Stage of Production: As specified immediately above in “Animals”.
Dose or Inclusion Rate: Approximately 0.5 to 10 mg L-thyroxine/kg diet or 200 to 285 g TIC/ton feed; 60 mg L-thyroxine implants in summer and autumn only not every season year round and with higher plane of nutrition for sheep producing wool; 150 mcg/kg body wt daily injection for mohair in goats; improvement for the cited uses is adding Mg at 5% to 300% of minimum diet requirement (essentially no or low excess Mg toxicity) because of close thyroid hormone and Mg relationship; coating for thermostability to survive steam pelleting.
Administration: Preferably in diet for growth, feed conversion, and feathering; for grazing animals may preferably administer implant for timed release; for show animals daily injections may be a desirable option.
Usefulness: Stimulates metabolism and heat production; in confinement need good ventilation system to remove excess body heat; must provide adequate or higher plane of nutrition to support additional lean growth; toxic if added substantially in excess; needs FDA and AAFCO approvals.

### EXAMPLE 4(a)

Use: Composition made up of a blend of trace minerals in organic form (that is, iodine, selenium, and zinc) at specific levels (that is, within designated ranges) mainly for the purpose of enhancing rate of feathering in avian species; also improves hair coat (hair growth) in mammals, mohair in angora goats, wool growth (fleece weight) in sheep; improves hoof health (reduces severity of hoof lesions) in horses, cows, sows, and sheep; improves fur coat (pelt) in fur bearing animals (e.g., mink and foxes); increases integument growth.
Animals: As specified immediately above in “Use”, including especially avian species (poultry and other birds) feathering; dairy cattle; breeding ewes and sows; horses.
Sex: Both sexes.
Age or Stage of Production: All young (non-adult), growing and feathering birds; show birds; birds raised for hackle feathers; adult poultry or other birds after the molting process when feather rejuvenation is occurring, and so on; dairy cattle and breeding ewes in particular; fur bearing animals such as mink and foxes grown for their pelts.
Dose or Inclusion Rate: Preferably, thyroactive iodinated casein (TIC) at about 100 to 720 g/ton feed, selenium yeast at about 0.1 to 0.3 mg/kg diet (0.3 mg/kg is the legal limit), which additive contains “selenomethionine”, and zinc methionine at about 20 to 100 mg Zn/kg feed; various combinations of levels of the 3 organic trace mineral compounds are acceptable; any 2 of the compounds constitute an effective blend as well; optionally, the vitamin biotin can be added for skin, foot, and hoof health at 5 mg/100 to 150 kg BW in horses, 20 mg/day for dairy cows, and 350 mcg/kg diet for breeding sows and further, a methionine source such as DL-methionine or methionine hydroxy analog (e.g., about 0.025% to 0.10%) can be added to improve feathering.
Administration: Preferred method administering the composition is in the diet, and it may be included in another premix such as a vitamin or trace mineral pre-mix to deliver the appropriate levels of inclusion to complete feed.
Usefulness: Each of the components of the proprietary feather growth enhancing composition are known to improve the rate of feathering in avian species; selenium has an FDA limit of 0.3 mg Se/kg of complete feed; thyroid hormones stimulate metabolism including feather growth, and selenomethionine and zinc methionine provide two forms of methionine that have been associated with more feather growth in poultry; some birds receiving the blend may not be going into the human food chain (e.g., for production of hackle feather for tying flies as fishing lures) which may affect the regulatory status or approval of the proprietary composition; skin condition including the ability to heal cuts and scratches is usually improved along with the better feather when zinc methionine is administered.

### EXAMPLE 5

Use: To reduce body fat and increase the proportion of lean tissue in poultry, especially meat-type birds that are overweight or have excessive body lipid content that are raised for processing or for replacements as breeding stock, or other animals that are too fat or obesely overweight (e.g., dogs), or show animals such as broilers, pigs, sheep, or beef cattle; caged laying hens with fatty liver syndrome; optionally, betaine at ˜800 to 1,000 mg/kg diet and/or L-carnitine (e.g., 90 mg/bird daily for pigeons) can be combined with thyroid hormone(s).
Animals: Broiler chickens grown to larger weights which tend to put on more abdominal fat (pad); broiler breeder replacement pullets (females); obese dogs or cats (usually spayed or neutered); show broilers, pigs, sheep, or beef cattle; caged laying hens with fatty liver syndrome.
Sex: Broiler chickens of both sexes but especially females with more body fat, less lean; female broiler breeder replacements; both sexes of dogs and cats especially spayed or neutered; both sexes of show birds or animals; adult caged laying hens.
Age or Stage of Production: Broiler chickens in the growing and finishing phases; broiler breeder replacement pullets in growing, developing, and prelay phases; adult dogs and cats; prior to market age for broilers, pigs, sheep, and beef cattle; caged laying hens diagnosed with fatty liver syndrome.
Dose or Inclusion Rate: L-thyroxine at 0.2 to 20 mg/kg depending on duration (shorter feed higher level), T3 at about 25% of T4, or both at T4 level (˜1:4.22 T3:T4, for example) preferably 1 to 3 mg/kg to observe initial response over a week or so; optionally, can combine with betaine at about 800 to 1,000 mg/kg diet and/or L-carnitine (e.g., 90 mg/bird daily for pigeons) can be combined with thyroid hormone(s); injected hormones can be given at ˜60% of oral dose.
Administration: Preferably in the diet for poultry and other food producing animals; tablets for pets orally; diet or daily or regular injections for show broilers, pigs, sheep, or beef cattle.
Usefulness: Stimulates metabolic rate (heat production) utilizing body fat stores; excessive doses can cause severe body weight loss; betaine spares methyl group requirement normally supplied by choline and methionine; L-carnitine is involved with fat transport and metabolism.

### EXAMPLE 6

Use: To increase the iodine content of meat from poultry, pigs, beef, sheep, or other animals, the iodine content of eggs (that is, table eggs or fertile eggs) from poultry species, and the iodine content of milk from lactating animals (mammals) by administering low levels of exogenous thyroid hormones (T3, T4, or both); optionally, in combination with inorganic iodides or iodates, or other sources of iodine such as seaweed.
Animals: Meat-type poultry or egg-laying strains of poultry; lactating animals such as dairy cows, beef cows, breeding ewes, sows, or dairy goats; meat-type food-producing animals such as pigs, beef cattle, sheep, goats.
Sex: Both sexes.
Age or Stage of Production: Preferably in the finishing phase prior to slaughter in meat-type poultry and other animals; during production of table eggs or fertile eggs; and during lactation.
Dose or Inclusion Rate: Preferably, about 4 mg inorganic iodide/kg diet or equivalent iodine from T4 (65.34% iodine by weight); iodine range about 0.1 to 10 mg/kg; T3 or T3 and T4 acceptable usually in about 1:4.22 T3:T4 ratio.
Administration: In the diet; pure iodine can be administered through the drinking water as well; excessive thyroid hormones can cause severe weight loss; enhancing meat, eggs, or milk with iodine can also be a side effect from other main benefits of exogenous thyroid hormones.
Usefulness: Elevating circulating and tissue thyroid hormone levels and/or iodine levels increases the iodine content of meat, eggs, and milk; care must be exercised not to exceed FDA limits for iodine in these products (for example, 150 mcg iodine per egg); can be sold as designer foods such as Eggland's Best Eggs with 70 mcg of iodine per egg from inorganic iodine and seaweed iodine.

### EXAMPLE 7

Use: To improve semen quality of poultry and other animals, mix thyroid hormone (that is, T3 preferably, T4, or both at physiological or higher level); add to semen to increase O2 consumption by spermatozoa, to improve fertility rate of eggs or conception rate in animals, and decrease embryonic mortality; optionally, in combination with one or more substances to improve sperm characteristics (such as L-carnitine, acetylcamitine, leutinizing hormone (LH), kallikrein (enzyme), theophylline, glutamic acid, glucose, blood serum, heparin, pGlu-Glu-ProNH2, 2′-deoxyadenosine, D-Penicillamine, polyvinyl alcohol, pentoifyllline, dibutryl cAMP, hypotaurine, and taurine) as reported in scientific literature); in semen collection (for example, getting semen from male turkeys, stallions, bulls, rams, boars, roosters, etc.) for use in artificial insemination; dose level in bulls—physiological T3 in semen about 0.1 ng/mL can be increased to 12.5 ng/mL or T4 from 1.2 to about 4.7 ng/mL (normally transferred from blood at lower rate than T3).
Animals: Fresh semen from adult males of all avian species or other animal species collected for use in artificial insemination.
Sex: Male.
Age or Stage of Production: Adult males in reproduction.
Dose or Inclusion Rate: Preferably add T3, T4, or both to achieve normal physiological levels or higher (for example, in bulls semen T3 is ˜0.1 ng/mL which can be increased to about 12.5 ng/mL or T4 is ˜1.2 ng/mL which can be increased to about 4.7 ng/mL as T4 is transferred from blood into semen at lower rate.
Administration: Direct addition to fresh semen or frozen semen that has been thawed.
Usefulness: Dose level for thyroid hormones depends on existing T3 and T4 in the semen and probably season of the year to get targeted normal or higher physiological levels in semen by difference for artificial insemination.

### EXAMPLE 8

Use: To increase milk yield in adult, female lactating mammals such as dairy cows, dairy goats, sows, ewes, dogs (bitches), mink, and other lactating animals by administration of thyroid hormones with dietary inclusion rate and duration of treatment depending on species; in combination with magnesium at 5% to 300% of requirement, preferably about 50% to 200%, to support oxidative phosphorylation enzymes, and optionally taurine (about 0.025% to 0.15% of diet which appears to help regulation of magnesium homeostasis); stimulation of basal metabolic rates requires higher plane of nutrition to help maintain normal body condition.
Animals: All mammals.
Sex: Adult females.
Age or Stage of Production: Adult female mammals during lactation; for relatively short lactations such as for sows (14-28 days) administer over the entire lactation whereas for long lactations such as for dairy cows administer in the declining phase of lactation.
Dose or Inclusion Rate: Preferably administered in the diet; for sows about 227 g TIC per kg complete feed; for dairy cows 0.5 to 1.5 g TIC per ton feed per 100 lb body weight in the declining phase of lactation and may cause sensitivity to heat (heat distress); in combination with magnesium (for example, 500 to 2,000 mg per kg complete feed), and optionally, taurine (about 0.025% to 0.15%); a higher plane of nutrition including protein, carbohydrates, lipids, minerals, vitamins, and water needed to help maintain normal body condition.
Administration: Preferably administered in the diet through inclusion of a premix containing the thyroid hormone(s) and magnesium, optionally with taurine.
Usefulness: Dietary thyroid hormones such as from TIC stimulate basal metabolic rate and body heat production resulting in increased milk yield during lactation; for breeding animals this improves the performance of the young nursing animals; supplemental magnesium assures adequate amounts to support enzymes involved in metabolism, and despite variable levels of magnesium intake by the lactating females, the supplemental magnesium provides enough yet with very low toxicity and excess magnesium is excreted in the urine and feces; taurine is supplied as the amino acid or other forms such as magnesium taurate.

### EXAMPLE 9

Use: To ameliorate or eliminate detrimental effects of disease challenges or metabolic disorders which diminish blood thyroid hormone levels by administering exogenous thyroid hormones to poultry and other animals to bring circulating levels toward normal or back to normal.
Animals: All animals including birds.
Sex: Both sexes.
Age or Stage of Production: All ages, stages of life, and phases of production.
Dose or Inclusion Rate: Thyroid hormones (usually T3 and T4) are administered to try to achieve blood physiological levels despite disease challenge and/or metabolic disorder, approximately 0.2 to 20 mg T4/kg diet, for example, or preferably equivalent TIC to provide both T3 and T4.
Administration: Thyroid hormones preferably administered by diet, or water if soluble product is available, or by injection though more labor intensive; injection or implantation are the most direct methods as they provide thyroid hormone(s) to the bloodstream or tissues by-passing intestinal digestion and absorption which may be impaired in disease conditions or metabolic disorders.
Usefulness: At least part or perhaps all of the lost performance which would have occurred as a result of low thyroid hormone levels may be recovered by administering the exogenous thyroid hormones.

### EXAMPLE 10

Use: To control, maintain, or restrict body weight of poultry or other animals, especially those designated to be grown for replacement birds or animals and need to meet body weight guidelines for better subsequent reproductive performance.
Animals: Poultry and other animals, especially meat-type birds or animals grown as replacements for reproductive purposes.
Sex: Both sexes, but mainly female replacements.
Age or Stage of Production: Primarily, during the growing and developing phases for replacement birds or animals; can be used for body weight control, maintenance, or restriction of avian or other animal species at near market ages or as adults.
Dose or Inclusion Rate: Low levels of exogenous thyroid hormones (e.g., 0.2 to 10 mg L-thyroxine/kg diet or use product with T3 and T4 such as TIC); T3 more effective than T4 at equal doses in lowering body weight.
Administration: Preferably in diet or drinking water.
Usefulness: Dose or inclusion rate of thyroid hormone(s) depends on mount of body weight targeted; T3 more effective than T4 at equal doses in lowering body weight (e.g., at 10 mg/kg diet each); caution in that excess exogenous thyroid hormone(s) can cause too much weight loss or possible death.

### EXAMPLE 11

Use: To accomplish reproductive quiescence and rejuvenation in adults of avian and other animal species by administering pharmacological levels of thyroid hormone(s) that decrease testosterone and cause transient inhibition or diminution of semen (sperm) production, and in some cases feather molt in avian males especially when given in conjunction with short day length, followed after treatment and photostimulation with improved semen production and fertility; administer both T3 and T4 preferably (e.g., 4 mg T3/kg and 16 mg T4/kg diet) or T3 or T4 individually; procedure could be termed “male molting” although feather loss may be less than in hens; L-thyroxine 2-20 mg/kg diet or TIC to supply T4 at these levels along with T3; and by continuous feeding low level of TIC (e.g., 0.022%) to adult males to help counteract the age related decline in fertility by favorably influencing male reproduction (sperm output); optionally, in combination with magnesium.
Animals: Avian species, especially meat-type chicken or turkey breeders which tend to lose libido and fertility with age and need a rest period or reproductive quiescence for rejuvenation to increase subsequent relative semen production.
Sex: Adult males, especially aged males that are declining in fertility.
Age or Stage of Production: Adult males that are advancing in age such that libido and fertility are substantially declining or expected to so; may be used in conjunction with a molting procedure for breeder hens (that is, short day length, low nutrient density feeds, culling of obviously unproductive or low fertility males as hens are culled).
Dose or Inclusion Rate: Approximately 4 mg T3/kg diet and 16 mg T4/kg diet, or separately at similar levels to induce reproductive rest in adult males; continuous feeding of TIC at about 0.022% of diet to breeder males in declining fertility for increased sperm output; optionally, in combination with magnesium (e.g., MgO) at 5 to 300% of minimum requirement.
Administration: Preferably through the diet or drinking water rather than by injection or implantation which are labor intensive.
Usefulness: Some loss of body weight is likely to occur; exogenous thyroid hormone(s) decrease testosterone as one of the modes of action; testes may enlarge; excessive T4 (25 mg/kg diet or more) has been shown to be toxic to broiler breeder males (death); testes shown to enlarge in broiler breeder males given 25 or 40 mg T4/kg diet.

### EXAMPLE 12

Use: To induce transient hypothyroidism in young males of avian and other animal species by administering a diet deficient in iodine, magnesium, and selenium (or as deficient as possible with available ingredients) and a goitrogenic substance (e.g., from natural feedstuff such as rapeseed meal or high gossypol cottonseed meal) at a critical age in testicular development to achieve subsequent increases in testes size and spermatogenesis and in persistency of fertility in these males as adults active in reproduction.
Animals: Adult poultry and other avian species, and other animal species, that are used for breeding stock.
Sex: Males of avian species, especially poultry, to be used for breeding stock; males of other animal species, especially food producing animals.
Age or Stage of Production: Adult reproductive stage, actively mating naturally or used as breeding stock for artificial insemination.
Dose or Inclusion Rate: Omit iodine, magnesium, and selenium for any dietary supplements; formulate diets with available ingredients lowest in these minerals; utilize natural antithyroid or goitrogenic substances in feedstuffs if possible or add substances that have these effects to create transient hypothyroidism at a critical time in testes development which time (age range) is species specific; for example, 0.5% sodium perchlorate in diet inhibits thryoid function; thiouracil or methimazole commonly used in rat and mice research not approved for feed; for example, 15% rapeseed meal or high-gossypol cottonseed meal are goitrogenic; it is desired to decrease circulating thyroid hormones.
Administration: Preferably in the diet or drinking water.
Usefulness: Helps counteract the natural decline in libido and fertility in avian and other animal species males by increasing adult testes size and sperm production as a result of transient hypothyroidism at a critical period in testicular development, testosterone levels are unchanged in adult treated or untreated poultry or other animals; also effective in male Tilapia fish; critical period in testicular development must be strictly adhered to for each species for procedure to be effective.

### EXAMPLE 13

Use: To protect poultry and other animals from overexposure to radioactive iodine, primarily 124-I through 131-I, which is taken up by the thyroid and causes tumors and other abnormalities, by: 1) temporarily administering potassium iodide, other iodine source, EDDI, or seaweed to provide about 10 mcg/100 cc serum by daily oral doses in feed or water for blockade and dilution effects against radioiodine, 2) in combination with docosahexaenoic acid (DHA, C22:6, n3) to provide about 100 to 300 microM levels in serum to enhance the inhibitory effect of iodide on thyroid follicle proliferation, or 3) instead of KI, administer sodium bromide (NaBr) at nor more than 50 to 100 mg/kg diet, and 4) optionally, along with KI+DHA or NaBr+DHA, administer exogenous thyroid hormones, preferably from a product containing both T3 and T4 (in about 1:4.22 ratio) such as thyroid powder or thyroactive iodinated casein, to help maintain normal metabolic function during the stress.
Animals: All avian species, especially poultry, that have a thyroid gland, and other animal species, including food-producing animals, zoo animals, and humans.
Sex: Both sexes.
Age or Stage of Production: All ages with doses appropriate for the metabolic body size of the bird or animal, with young being administered lower and adults higher doses within the acceptable ranges.
Dose or Inclusion Rate: For humans, the KI doses in tablet form are well defined—130 mg for adults 18-40 yrs including pregnant and lactating women; 65 mg for children and adolescents 3 through 18 yrs; 32 mg for children 1 month through 3 yrs; 16 mg for newborn to 1 month; prophylactic effect lasts 24 hrs so daily dosing is required; adults over 40 yrs only need to take KI in case of large (>500 cGy) internal radiation dose to the thyroid to prevent hyperthyroidism; docosahexanaenoic acid (DHA, C22:6, n3) to provide about 100 to 300 mM concentration in serum; sodium bromide (NaBr) not more than 50 to 100 mg/kg of diet.
Administration: Preferably tablets to pets or companion animals, or humans; as dietary supplement to avian and animal species for food production, zoo animals and birds, fish and certain other farmed aquaculture species such as eels.
Usefulness: Bromine can replace iodine on the 5 position of both T3 and T4 with no loss of thyroid hormone activity; radioactive iodine uptake is blocked by the presence of excess iodine and/or bromine (that is, iodine, bromine, or both); DHA, an omega fatty acid, inhibits thyroid follicle proliferation and prevents goiter; thyroid hormones at normal physiological levels provide a safety factor during the stress.

### EXAMPLE 14

Use: To increase basal metabolic rate and body heat production in poultry hatchlings, nursery pigs, and other neonates during brooding or cool stress to reduce morbidity and mortality, and improve live performance overall by administering exogenous thyroid hormone T3, T4, or both at relatively low level in the diet or drinking water.
Animals: Avian species, especially poultry hatchlings or birds being brooded; nursery pigs; young calves, especially those housed outdoors in cool weather, and so on; adult animals housed in cool or cold conditions.
Sex: Both sexes.
Age or Stage of Production: Young birds during placement and brooding, especially those brooded under lower than optimal temperatures (e.g., 88 deg F. vs 94 deg F. to save on fuel expense; nursery pigs; other neonates in cool stress; adult birds or other animals in open housing in winter or outdoors.
Dose or Inclusion Rate: In the diet, T3 at 0.2 to 3 mg/kg or T4 at 0.2 to 12 mg/kg, or both; in the drinking water at about 56% of the level in feed.
Administration: Preferably in feed or water.
Usefulness: Under cool stress, the young or adult bird or animal responds by increasing blood thyroid hormone levels; however, this response may be delayed exposing the animals to temporary stress or may be insufficient so exogenous thyroid hormones can be helpful in these conditions.

### EXAMPLE 15

Use: To ameliorate the effects of heat stress in avian species, especially poultry, and other animals in growing, finishing, and adult stages of life by providing low levels of exogenous thyroid hormones which are diminished in the circulation as a result of high ambient temperatures, in order to improve productive performance and reproduction; optionally, in combination with L-carnitine (e.g., 90 mg/bird daily in pigeons) and/or Salicylic acid (e.g., 0.9 to 1.75 fluid oz. Unisol with 460 g/quart per 1,000 lbs bird or animal body weight).
Animals: All species.
Sex: Both sexes.
Age or Stage of Production: Poultry and other animals in the growing, finishing, and adult stages which are more susceptible to heat stress than young avians or other animals.
Dose or Inclusion Rate: Low levels of T3 and T4 in ratio of about 1:4.22 is preferred in feed (e.g., 0.1 to 1 mg T3/kg or 0.4 to 4 mg T4/kg, or both); or in water at lower levels such as about 56% of level in feed; TIC at 0.011% or 0.017% shown to be helpful to cockerels in heat stress for improving general semen characteristics; L-carnitine at 90 mg/bird daily to pigeons reduced heat production during heavy exercise; aspirin or aspirin-like compounds such as salicylic acid are vasodilators and increase free T4 in blood, inhibit formation of T3 from T4.
Administration: Preferably in feed, water, or both heat stress (that is, panting in poultry or other animals), usually defined as above 90 deg F. and 50% relative humidity.
Usefulness: Although it may be counterintuitive, thyroid hormones are administered at low levels in heat stress because they are involved in many metabolic functions in the animal body even though they themselves generate some body heat production as well; high ambient temperatures decrease the circulating levels of thyroid hormones and cold weather increases them.

### EXAMPLE 16

Use: To increase and sustain egg production and improve egg shell quality in avian species, especially poultry producing table eggs or fertile hatching eggs by administering 0.01 to 0.04% TIC or other substances containing T3 and T4; optionally, magnesium can be added at 5 to 300% of minimum requirement to support increased metabolic rate, Calsporin (Bacillus subtilis C-3102 spores), lactic acid (about 0.5% of diet), and/or Hy-D (25-hydroxy-cholecaliferol, an active form of vitamin D3) at 37 to 69 mcg/kg diet for egg shell quality improvement.
Animals: Avian species, particularly adult females in egg production.
Sex: Females only.
Age or Stage of Production: Adults in egg producing (e.g., caged laying hens producing table eggs or breeding ducks producing fertile eggs).
Dose or Inclusion Rate: TIC in diet at 0.01 to 0.04% to provide T4 and T3, or approximate equivalent as thyroid powder or rendered product; optionally, magnesium (e.g., MgO) to support heightened metabolic rate, Calsporin (Bacillus subtilis C-3102) spores with patent for improving egg shell quality at 0.003% of diet), Hy-D 25-hydroxycholecalciferol an active form of vitamin D3 at about 20 to 69 mcg/kg diet, and/or lactic acid.
Administration: Preferably in the feed during egg production.
Thyroid hormones at relatively low levels of inclusion in feed (TIC at 0.01 to 0.04%) can improve egg production; optionally, magnesium can be added to support higher metabolic rate, and egg shell enhancing supplements individually or combined can be added (e.g., Calsporin—Bacillus subtilis C-3102 spores, Hy-D 25-OH-vit D3), lactic acid at about 0.5%).

### EXAMPLE 17

Use: To increase bone breaking strength in caged laying hens near the end of the egg production cycle and prior to slaughter in order to strength bones for handling the birds and prevent broken, shattered, and splintered bones by administration of 0.062% TIC for 1 wk prior to processing the spent hens; can be administered to aged males for improving bone strength as well; optionally, magnesium 5 to 300% of minimum requirement and/or Hy-D 25-hydroxy-vitamin D3 at 20 to 69 mcg/kg diet.
Animals: Aged adult fowls, especially those in cages which tend to have weaker bones than those on litter or slats.
Sex: Both sexes, but primarily females.
Age or Stage of Production: Near the end of the final cycle of egg production when hens are to be slaughtered and replacements brought in; males may be treated as well to increase bone strength.
Inclusion rate of about 0.062% TIC in diet for about 1 wk; optionally, magnesium at 5 to 500% of minimum requirement, and or Hy-D 25-hydroxy vitamin D3 at about 20 to 69 mcg/kg diet; TIC 0.062% to 0.249% in layer diets (3% Ca) increased bone strength in linear fashion.
Administration: Preferably in feed, but T3 and T4 both can be administered in the water
Usefulness: Increasing bone strength over a 1 wk period is the objective, and 25-OH-vitamin D3 is an active metabolite which complements TIC in this application; magnesium supports higher level of metabolic activity.

### EXAMPLE 18

Exogenous thyroid-active substances—T4 (3,5,3′,5′-tetraiodo-L-thyronine), T3 (3,5,3′-triiodo-L-thyronine), thyroprotein, thyroactive iodinated casein, thyroid hormone, thyroid (thyroglobulin, thyroidine, proloid), L-thyroxine (levothyroxine), T3 (liothyronine, tertroxin, cytomel); subtances that stimulate the thyroid to produce T4 or T3 can be administered, including TSH (thyroid-stimulating hormone, thyrotropin, thyrotropic hormone, thytopar, ambinon, or Dermathycin trademark), TRH (thyrotropin-releasing hormone), and the like; thyroactive iodinated casein is preferred for many feed applications, T4 specifically for molting adult female fowls; defatted, dessicated animal thyroid powder or rendered animal thyroid tissue (as apparently allowed in AAFCO animal byproduct meal definition) with preferably 50% to 95% thyroid tissue included.

### EXAMPLE 19

Proprietary rendered animal byproduct meal containing 1 to 100% poultry or animal thyroid tissue, preferably 50 to 95% as apparently allowed by current AAFCO feed ingredient definition of animal byproduct meal (assuming animal indicates either poultry or other animals as in certain other definitions though undefined in said definition); defatted, desiccated bovine or porcine thyroid tissue from USDA inspected slaughter plants is used for humans (and their pets); porcine thyroid powder has about 0.21 to 0.25% T4 plus T3 and about 0.19% T4 content, and there is about 1 g thyroxine per 600 g defatted (<5% fat) and dessicated porcine thyroid powder; such products can provide T3 and T4 for administering exogenous thyroid hormone to avian and other animal species.

### EXAMPLE 20

Substances to enhance, improve, or potentiate the action, effect, or response of exogenous thyroid hormone(s) are provided:
(a). Magnesium, a macromineral nutrient, required by over 300 enzymes especially important in oxidative phosphorylation involved with enhanced metabolic rate; because of low toxicity, Mg can be added with exogenous thyroid hormones to complement them regardless of the variation in dietary magnesium (unless very high already), and excess will be excreted in the feces and urine, help maintain tissue and blood levels adequate when administering thyroid hormone(s); sources include MgO, MgCl2, MgSO4, KSO4-MgSO4, magnesium taurate; exogenous thyroxine hormone(s) decrease blood Mg by driving it into tissues and Mg supplementation assures adequate blood and tissue levels; in present invention, Mg is suggested as an improvement over previous patent claims and embodiments (e.g., growth, feathering, egg production increases with TIC); 5% to 300% of minimum Mg requirement is administered in combination with thyroid hormone(s); few adverse effects with excessive Mg intake but include decreased blood Ca and K; about 1% Mg in body found in blood; cold increases blood thyroid hormones and Mg requirement, heat decreases thyroid hormones and maybe Mg needs.
(b) Taurine appears to be important in Mg homeostasis in the body lowering blood pressure, binding Ca, stabilizing platelets and so on; apparently specific action as “Mg-sparing” parathyroid hormone (that is, gamma-L-glutamyl taurine); administer at about 0.025 to 0.15% of diet with or without Mg to support increased metabolic rate due to T3, T4.
(c) Sodium salicylate increases the free dialyzable form (from protein bound form) of T4 in blood making it more readily available; however, it inhibits to some extent the conversion of T4 to T3; especially in situations in which T4 is the preferred exogenous thyroid hormone, sodium salicylate may potentiate the effect by increasing availability of the free form of T4 ready to use; aspirin and salicylic acid are other related compounds; Unisol is a liquid commercial product containing 460 g sodium salicylate per quart, administered to 1,000 lb bird or animal weight at 0.9 to 1.75 fl oz.
(d) Protease enzymes can be used to increase the digestibility of the protein component of a supplement or diet, including thyroprotein, thyroid powder, or rendered thyroid tissue to hydrolyze the iodinated thyronines containing iodine and increase the thyroid hormone effect; proteases sometimes designated as peptidases, proteinases, peptide hydrolases, or proteolytic enzymes; exo-type hydrolyses peptides starting at either end thereof or endo-type that acts internally in polypeptide chain (endopeptidases); inclusion rate in diets depends on protease manufactures recommendation, and can be added to thyroid hormone containing substance (admixture).
(e) L-carnitine is useful in pigeons to improve fatty acid oxidation during heavy exercise and to reduce heat production (90 mg/pigeon for 1 wk) (Janssens et al., 1998); Buyse et al. (2001) L-carnitne at 100 mg/kg diet elevated proportional and absolute heart weight (not due to right ventricle hypertrophy) potentially beneficial for ascites susceptible birds, and increased blood T3, decreased abdominal fat in female broilers; Owen et al. (1994) 40 mg/kg diet improved pig weight gain, feed/gain, muscle, and lowered backfat thickness (Netherlands study cited); Anonymous (1998 Int. Pig Topics) 50 mg per boar daily yielded one additional sow insemination per ejaculate; Baumgartner and Blum (1998) 30-50 mg/kg diet for 4 wk weaned pigs best, but the higher level better for earlier weaning, boars 100-200 mg/kg diet, sows in gestation and lactation 50 mg/kg diet, milk replacers for pigs 500 mg/kg diet; Rabie et al. (1997) 50-500 mg/kgdiet increased egg interior quality and % white but decreased % yolk; Up to 1,000 mg/kg diet to pigs.
(f) Hy-D, which is 25-hydroxy-cholecalciferol, an active form of vitamin D3 is useful for increasing egg shell quality and bone strength by eliciting production of calcium binding protein in the intestine to absorb calcium, phosphorus, and Mg; typical level of inclusion is 69 mcg/kg diet, useful range 20 to 69 mcg/kg diet.
(g) Melengestrol acetate (MGA) is a progestin that successfully causes the regression of ovary and oviduct in laying fowl and allows complete recovery when treatment is removed; 4 or 8 mg/hen/d reported effective; however, it takes abt 10-15 mg/hen/day for complete cessation of egg production (Koch et al., 2005).
(h) Zinc compounds such as ZnO, Zn sulfate, or Zn acetate can be used to induce the regression of reproductive tract in laying fowl; a level of 0.25 to 1.00% ZnO is administered with thyroid hormones to complement the T4 molt treatment; zinc in excess of requirements may have a direct inhibitory effect on progesterone production in F1 granulosa cells of the ovary (Johnson and Brake, 1992), and Zn level used was 2,800 ppm.
(i) Iodolactones and/or iodoaldehydes can correct or inhibit thyroid enlargement (goiter). Stimulation of porcine thyroid follicles in vitro with 2 and 20 microM iodide rapidly induced aptosis (4-6%) comparable to about 40-fold lower doses of 0.05 to 0.5 microM delta-iodolactone (Langer et al., 2003), and it is noted that high iodine doses cause thyroid atrophy but only to normalization of thyroid size; Paneels et al. (1996) 2-iodohexadecanal is a major iodolipid in thyroid tissue; Dug-rillon and Gartner (1992) isolated porcine thyroid follicles treated with 100 and 300 microM concentrations of docosahexaenoic acid (DHA, C22:6, n3) enhanced the inhibitory effect of 10 microM iodide on thyroid follicle proliferation.
(j) Betaine at 800 to 1,000 mg/kg diet elevates T4 and T3 in blood of pigs and increases growth rate and decreases feed conversion ratio (ZiRong et al., 1999); betaine improves breast meat yield in poultry at 0.10-0.15% of diet; spares methionine and choline methyl donor functions; helps potentiate coccidiostats by preventing invasion of coccidia into intestinal epithelium; decreases FCR and backfat, and increases loin eye muscle in pigs. Zabaras-Krick (1997), level was Betafin at 1-1.25 kg/tonne and Betafin has 97% betaine; Remus (2001) reported the breast meat yield improvements in broilers and turkeys with 1,000 mg betaine/kg diet.
(k) Calsporin (Bacillus subtilis C-3102 spores at 0.003% of diet), Calpis USA, Inc., Elgin, IL is a patented feed additive for improving egg shell thickness; mode of action is presumably that increased lactic acid production improves limestone digestibility; and/or with lactic acid (e.g., about 0.5% of hen diet) directly.

### EXAMPLE 21

A variety of methods provide, through hydrolysis of iodinated proteins, iodinated peptides (hyrdolyzed proteins). Older manufacturing methods used casein; newer ones start with L-tyrosine.

### EXAMPLE 22

Phenyalanyl residues in casein are converted to tyrosyl residues. 1% thyroxine content in thyroactive iodinated casein is typical, but Phe to Tyr gives 4.5% Phe (now Tyr) plus 5% Tyr for higher yield.

### EXAMPLE 23

Other sources of phenyl groups spare Tyr in casein for full potential yield of thyroxine. Tyr+Tyr in Casein causes loss of some valuable Tyr which become Ala; combine Tyr in casein with DIT or Diiodophenyl-pyruvic sources of phenyl group to increase yield of thyroxine.

### EXAMPLE 24

The combined or serial use of any two or three methods described above in the previous three examples. Peptides are commercially available; Phe (4.5%) to Tyr and DIT or other phenyl groups sources increase yield up to 17.8% thyroxine in iodinated casein when now 1% (from 5% Tyr).

### EXAMPLE 25

Coating thyroxine or thyroxine containing compounds or mixing them with certain excipients or compounds increases thermostabilility through steam pelleting, to improve shelf-life in storage, to obtain granular or micro-granular physical forms, for better handling, mixing, flowability, add color, impart flavor, less dustiness. Coating for thermostability is useful for pelleted and crumbled feeds to retain potency of the product; altering physical form to granule or microgranule improves mixing and handling characteristics; certain excipients extend shelf-life to 6 months or more.

The present invention and any related thereto provide thyroxine (T₄) as natural molting hormone for avian species. Research was designed to provide the commercial egg industry with a “hen-friendly” induced molting program, that will satisfy animal welfare considerations, by dosing hens with L-thyroxine. The following experiments, 1 through 4, were made possible by a $20,000 grant from United Egg Producers and were conducted with caged laying hens (chickens): 1) to validate the concept that adequately increasing circulating thyroxine (T₄) can induce molting, 2) to determine the optimum dose, and 3) to evaluate effectiveness of different thyroxine sources. Other experiments were subsequently carried out with broiler breeder hens and roosters, caged laying hens, and turkey breeder hens to evaluate the responses of other breeds and classes of poultry to the “T₄ molt” to accomplish reproductive rejuvenation.

Experiment 1. Confirmation that Injected Thyroxine Induces Molting. The first study with 60-week old Hy-Line W-36 White Leghorn hens, not previously molted, lasted 40 days and was designed to confirm the efficacy of injecting T₄ from Na-L-thyroxine pentahydrate intramuscularly as a trigger for molting and cessation of egg production. While acknowledging that injecting individual hens is commercially impractical (Webster, 2003), nevertheless an initial study was needed to establish the efficacy of T₄ when it is delivered directly into the hens in precisely measured doses. The photoperiod was 17 hours of light per day (0330 hours to 2030 hours).

Egg production in the Saline Group remained unchanged throughout the injection and post-injection intervals (day 15 to day 40 inclusive), and injecting 250 μg T₄ per kg body weight for 12 consecutive days did not reduce egg production significantly. Egg production was significantly reduced 4 days after the start of injecting the 500 and 1,000 μg T₄ Groups, with egg production ceasing entirely in the 1,000 μg T₄ Group by the 8^{th} day of T₄ injection. A week after injections were terminated, several hens in the 500 μg T₄ Group resumed sporadic egg production whereas hens in the 1000 μg T₄ Group did not resume production for the remainder of the experiment. Injecting 2,000 and 4,000 μg T₄ per kg body weight for 3 consecutive days triggered a rapid and complete cessation of egg production within 6 or 4 days, respectively, which did not subsequently recover for the remainder of the experiment.

Two hens (2 of 8=25%) in the 1,000 μg T₄ Group died on the 9^{th} and 10^{th} day after the start of T₄ injection, and one hen (1 of 4=25%) in the 4000 μg T₄ Group died on the 8^{th} day after the start of T₄ injection. No mortality occurred in the remaining Groups throughout the experiment. None of the hens in the Saline group molted, and three hens in the 250 μg T₄ Group began to molt 10 to 15 days after the start of injections. In the 500 and 1,000 μg T₄ Groups, molting began in all cages on the 11^{th} and 9^{th} days, respectively, after the start of T₄ injection. For the 2,000 and 4,000 μg T₄ Groups, molting commenced in all cages on the 9^{th} day after the start of injection. In all Groups, molting hens shed virtually all feathers within 7 to 10 days, and during the subsequent week feather regrowth progressed equally well in all Groups.

Body weights did not differ among the Groups prior to the injections, and the Saline Group retained the same body weight throughout the experiment. However, T₄ injections significantly reduced the body weight of all Groups. Complete cessation of egg production was associated with a 15 to 25% reduction in body weight at the onset of molt, a percentage that includes the weight of feathers lost. There was an inverse relationship between the T₄ injection dose and daily feed intake, with feed intake being significantly lower in hens injected with ≧500 μg T₄ when compared with the Saline-injected controls. The sole behavioral observation in Groups receiving T₄ was that higher doses (≧1,000 μg T₄ per kg BW per day) caused hens to be more excitable and “flighty” when taken from their cages for injections. Otherwise, no cannibalism or aggression was noted within or between cages. Once molting began the hens became less active and tended to remain sitting in their cages when humans entered the chamber.

Necropsies were conducted on the three birds that died (two from the 1,000 μg T₄ Group, one from the 4,000 μg T₄ Group) as well as four uninjected control hens, two hens from the 250 μg T₄ Group, two hens from the 500 μg T₄ Group, one hen from the 1,000 μg T₄ Group, and one hen from the 4,000 μg T₄ Group. There was no evidence that the repeated injections had damaged the breast muscle. Hens from the uninjected Control Group and 250 μg T₄ Group were well fleshed, had ample (Control Group) or appeared to have slightly reduced (250 μg T₄ Group) amounts of body fat, fully functional reproductive tracts, and ovaries containing typical hierarchies of 3 to 5 maturing follicles. A hard-shell egg was found in the shell gland of one hen from the 250 μg T₄ Group. Both hens in the 500 μg T₄ Group were molting, and their body fat was obviously reduced when compared with the Control and 250 μg T₄ hens. Both hens in the 500 μg T₄ Group had functional reproductive tracts including the presence of a partially calcified egg in the shell gland of one hen. The ovaries of both hens from the 500 μg T₄ Group had hierarchies of 3 or 5 maturing follicles. Hens in the 1,000 and 4,000 μg T₄ Groups were extremely lean, had completely regressed reproductive tracts (≦50% normal size) and ovaries containing deteriorating (<4 mm diameter) or fully regressed/immature (≦2 mm diameter) follicles. No obvious differences in thyroid sizes were observed among the Groups, the air sacs were clear in all hens examined, and no evidence of osteoporosis was detected.

TABLE 2
Hen-day egg production (%) beginning on day 14 at 2-day intervals by treatment;
L-thyroxine administered by i.m. injection beginning on day 15 (for 12 days at
250 mcg level, 8 days at 500 or 1,000 mcg levels, and 3 days at 2,000 or 4,000
mcg levels) (Experiment 1).

| Dose, mcg/kg | Day | | | | | | | | |
|---|---|---|---|---|---|---|---|---|---|
| Body Wt | 14 | 16 | 18 | 20 | 22 | 24 | 26 | 28 | 30 |
| 0 (Saline) | 75.0 | 85.4 | 95.8 | 72.9 | 89.6 | 93.8 | 83.3 | 77.1 | 100.0 |
| 250 (12 d) | 93.8 | 87.5 | 68.8 | 56.3 | 25.0 | 31.3 | 37.5 | 31.3 | 37.5 |
| 500 (8 d) | 87.5 | 93.8 | 31.3 | 31.3 | 6.3 | 6.3 | 6.3 | 6.3 | 25.0 |
| 1,000 (8 d) | 81.3 | 68.8 | 31.3 | 12.5 | 0.0 | 0.0 | 0.0 | 0.0 | 0.0 |
| 2,000 (3 d) | 62.5 | 87.5 | 87.5 | 75.0 | 50.0 | 12.5 | 0.0 | 0.0 | 0.0 |
| 4,000 (3 d) | 62.5 | 87.5 | 100.0 | 87.5 | 25.0 | 0.0 | 0.0 | 0.0 | 0.0 |

Note:
There were 4 cages of 2 or 3 laying hens each per treatment group.

TABLE 3
Body weight, body weight change, and feed consumption by treatments (Experiment 1)

| Dose, mcg/kg | Initial Body | End of Injections | Body Weight | Body Weight | Feed Intake, |
|---|---|---|---|---|---|
| Body Wt | Weight, g | Body Weight, g | Change, g | Change, % | g/hen/day¹ |
| 0 (Saline) | 1448 | 1448 | 0 | 0 | 86.2 |
| 250 (12 d) | 1495 | 1300 | 195 | 13 | 58.7 |
| 500 (8 d) | 1472 | 1299 | 173 | 11 | 52.3 |
| 1,000 (8 d) | 1513 | 1167 | 347 | 23 | 31.4 |
| 2,000 (3 d) | 1510 | 1234 | 277 | 18 | 20.2 |
| 4,000 (3 d) | 1387 | 1165 | 222 | 16 | 11.7 |

¹Feed consumption was measured from day 22 to day 28; see previous Table for injection days.

Experiment 2. Confirmation that Thyroxine Added to the Feed Induces Molting. The second study involved 102-week old Hy-Line W-36 White Leghorn hens (previously molted at 55 weeks old), lasted 30 days, and was designed to provide hens with T₄ (from Na-L-thyroxine pentahydrate) in the feed at sufficient levels to induce molting (e.g., loss of primary “flight” feathers), complete cessation of egg production, and full regression and involution of the reproductive tract. The photoperiod was 17 hours of light per day (0330 hours to 2030 hours).

The objective was to use T₄ to humanely induce molting in hens that are continuously provided with ad libitum access to palatable feed meeting or exceeding all National Research Council (1994) standards. Developing a fully efficacious yet affordable molting protocol was predicated on determining the minimum effective level for T₄ supplementation. Factors that potentially may affect the required level of T₄ supplementation include: (1) uncertainty regarding the efficiency of T₄ absorption by the gastrointestinal tract, (2) the possibility that continuous dietary ingestion of T₄ could trigger substantially different biological responses when compared single daily injections, and (3) the likelihood that daily T₄ intake would diminish in parallel with molt-related reductions in feed intake associated with cessation in egg production. A spontaneous and voluntary loss of appetite (anorexia) commonly accompanies seasonal molting and broodiness in a variety of avian species (Berry, 2003; Webster, 2003). Accordingly, the responses of hens to diets containing 10, 20, and 40 mg T₄/kg, to bracket the anticipated range of T₄ needed to cause an effective molt, were determined.

Egg production by the Control hens remained unchanged in both Chambers (i.e., exposed to either 6 or 10 days on test diets) throughout the 30-day experiment. Feeding 20 and 40 mg T₄/kg consistently reduced egg production within 4 days, whereas the 10 mg T₄/kg diet reduced egg production significantly only in Chamber 5 (6 days on test diets) but not in Chamber 6 (10 days on test diets). Removal of the test diets after 6 days caused sporadic egg production to resume at levels that were not lower than those of the Control group by day 18 in the 10 mg T₄/kg Group, and by day 20 in the 20 and 40 mg T₄/kg Groups, whereas feeding the 40 mg T₄/kg diet for 10 days caused egg production to cease completely for the duration of the experiment. No mortal-ity occurred in any of the Groups throughout the experiment. None of the hens in the Control group molted, half of the hens in 10 mg T₄/kg Group in Chamber 6 (10 days on test diets) began to molt 11 days after T₄ feeding was initiated, and hens in the 20 and 40 mg T₄/kg Groups in both chambers molted 9 to 11 days after T₄ feeding was initiated. In Chamber 6 (10 days on test diets) the hens fed 40 mg T₄/kg shed virtually all feathers within 7 to 10 days, and feather regrowth during the subsequent week progressed well. Behavioral changes were not apparent in molting hens, regardless of the test diet or Chamber. No cannibalism or aggression was noted within or between cages of birds. The hens became sedentary after feather loss began.

The Control Groups in both Chambers retained their initial body weight throughout the experiment. All T₄ test diets caused progressive reductions in body weight, with absolute body weight tending to return toward the initial values after cessation of feeding the 10 and 20 mg T₄/kg diets. In the 40 mg T₄/kg Group both the body weight and percentage change in body weight consistently remained depressed until the end of the experiment. Reduction in the absolute hen-day feed intake and in the percentage change in hen-day feed intake paralleled the respective contemporaneous change in absolute body weight and percentage change in body weight. Thus, hens fed the 40 mg T₄/kg test diet for 10 days completely ceased egg production, shed virtually all of their feathers, reduced their feed intake by approximately 85%, and lost approximately 21% of their initial body weight. The percentage shell values did not change over time in the Control Group, but were similarly reduced within 4 days after the start of feeding the 10, 20, and 40 ppm T₄ test diets. Whole egg weights did not change during the 4 day period, averaging 65±1, 62±2, 65±2, and 65±3 g (mean±SEM) for the Control and 10, 20, and 40 ppm T₄ Groups, respectively. Necropsies were conducted on 12 hens that had entirely ceased egg production after being fed the test diets. Two hens appeared to be coming back into production because small (3 to 5 mm diameter) follicles were developing although the oviduct was fully regressed. The remaining hens were extremely lean, had completely regressed reproductive tracts (≦50% normal size) and ovaries containing fully regressed and immature (≦1 mm diameter) follicles.

TABLE 4
Hen-day egg production (%) beginning on day 4 at 2-day intervals by
treatment; L-thyroxine administered in the diet at 0, 10, 20, 40 mg/kg
on day 5 for either 6 or 10 days by room (Experiment 2).

| Dose, | | | | | | | | | | | | | | |
|---|---|---|---|---|---|---|---|---|---|---|---|---|---|---|
| mg/kg | Day | | | | | | | | | | | | | |
| of feed | 4 | 6 | 8 | 10 | 12 | 14 | 16 | 18 | 20 | 22 | 24 | 26 | 28 | 30 |
| 0 (6 d) | 83 | 67 | 75 | 67 | 83 | 92 | 75 | 83 | 67 | 92 | 75 | 83 | 67 | 75 |
| 10 (6 d) | 75 | 58 | 25 | 8 | 17 | 0 | 33 | 25 | 58 | 75 | 83 | 42 | 83 | 75 |
| 20 (6 d) | 94 | 69 | 17 | 0 | 17 | 14 | 33 | 39 | 47 | 64 | 64 | 78 | 89 | 61 |
| 40 (6 d) | 67 | 42 | 8 | 0 | 0 | 0 | 0 | 8 | 25 | 25 | 50 | 17 | 33 | 33 |
| 0 (10 d) | 92 | 92 | 92 | 67 | 83 | 92 | 75 | 75 | 75 | 67 | 67 | 92 | 83 | 75 |
| 10 (10 d) | 58 | 50 | 42 | 17 | 17 | 17 | 17 | 25 | 17 | 33 | 50 | 25 | 25 | 33 |
| 20 (10 d) | 75 | 58 | 8 | 8 | 0 | 0 | 0 | 8 | 8 | 17 | 17 | 17 | 33 | 25 |
| 40 (10 d) | 67 | 33 | 8 | 8 | 0 | 0 | 0 | 0 | 0 | 0 | 0 | 0 | 0 | 0 |

Note:
There were 3 cages of 2 hens each per treatment group.

TABLE 5
Body weight (BW) and body weight change (% BWC) from day 1 by thyroxine
treatments, day 7 to day 30 (Experiment 2).

| Dose, | | | | | | | | | |
|---|---|---|---|---|---|---|---|---|---|
| mg/kg | Day 1 | Day 7 | | Day 10 | | Day 14 | | Day 30 | |
| of feed | BW, g | BW, g | % BWC | BW, g | % BWC | BW, g | % BWC | BW, g | % BWC |
| 0 (6 d) | 1532 | 1561 | 1.9 | 1529 | −0.2 | 1554 | 1.4 | 1586 | 3.5 |
| 10 (6 d) | 1501 | 1377 | −8.3 | 1293 | −13.9 | 1424 | −5.1 | 1483 | −1.2 |
| 20 (6 d) | 1582 | 1434 | −9.4 | 1373 | −13.2 | 1491 | −3.9 | 1521 | −3.9 |
| 40 (6 d) | 1629 | 1443 | −11.4 | 1350 | −17.1 | 1396 | −14.3 | 1481 | −9.1 |
| 0 (10 d) | 1421 | 1406 | −1.1 | 1421 | 0.0 | 1412 | −0.6 | 1426 | 0.4 |
| 10 (10 d) | 1477 | 1372 | −7.1 | 1335 | −9.6 | 1314 | −11.0 | 1400 | −5.2 |
| 20 (10 d) | 1680 | 1533 | −8.8 | 1431 | −14.8 | 1373 | −18.3 | 1448 | −13.8 |
| 40 (10 d) | 1572 | 1392 | −11.5 | 1309 | −16.7 | 1245 | −20.8 | 1322 | −15.9 |

Note:
Thyroxine treatment was added to diets on day 5.

TABLE 6
Feed consumption (FC, g/hen/day) and feed consumption change (% FCC) from days
2-4 by thyroxine treatments, including days 5-7, 8-10, and 11-14 (Experiment 2).

| Dose, | | | | | | | |
|---|---|---|---|---|---|---|---|
| mg/kg | Days 2-4 | Days 5-7 | | Days 8-10 | | Day 11-14 | |
| of feed | FC, g/hen/d | FC, g/hen/d | % FCC | FC, g/hen/d | % FCC | FC, g/hen/d | % FCC |
| 0 (6 d) | 100.7 | 97.7 | −3.0 | 88.7 | −11.9 | 98.0 | −2.7 |
| 10 (6 d) | 101.3 | 60.0 | −40.8 | 35.3 | −65.2 | 81.3 | −19.7 |
| 20 (6 d) | 103.0 | 42.0 | −59.2 | 33.7 | −67.3 | 73.7 | −28.4 |
| 40 (6 d) | 98.3 | 36.7 | −62.7 | 13.7 | −86.1 | 46.7 | −52.5 |
| 0 (10 d) | 108.3 | 101.3 | −6.5 | 91.0 | −16.0 | 102.0 | −5.8 |
| 10 (10 d) | 84.0 | 56.0 | −33.3 | 33.7 | −59.9 | 47.3 | −43.7 |
| 20 (10 d) | 120.7 | 54.7 | −54.7 | 30.0 | −75.1 | 32.7 | −72.9 |
| 40 (10 d) | 116.3 | 37.3 | −67.9 | 22.0 | −81.1 | 19.3 | −83.4 |

Note:
Thyroxine treatment was added to diets on day 5.

TABLE 7
Percent shell on eggs from thyroxine treatments; both rooms combined
because of the limited number of eggs in some groups (Experiment 2).

| | |
|---|---|
| Dose, mg/kg | Egg Shell, % (washed, dried) |

| of feed | Days 2 to 4 | Days 5 to 6 | Days 7 to 8 |
|---|---|---|---|
| 0 (6 & 10 d) | 8.20 (n = 12) | 8.00 (n = 17) | 8.38 (n = 25) |
| 10 (6 & 10 d) | 7.93 (n = 6) | 7.39 (n = 9) | 6.63 (n = 8) |
| 20 (6 & 10 d) | 8.38 (n = 10) | 8.08 (n = 13) | 5.56 (n = 2) |
| 40 (6 & 10 d) | 8.34 (n = 6) | 7.82 (n = 6) | 6.63 (n = 3) |

Note:
Number of eggs sampled is n.
Low calcium intake associated with low feed consumption for thyroxine-treated diets may largely be responsible for differences in egg shell %.

Experiment 3. Reducing the Photoperiod Minimally Enhances Molting Caused by Thyroxine Added to the Feed and Allows Response to Photostimulation Later. The third study was conducted with 96-week old Hy-Line W-36 White Leghorn hens (previously molted at 80 weeks of age) for 29 days to evaluate potential interactions between supplementing the feed with T₄ and reducing the photoperiod (8 hr vs 17 hr of light per day). The photoperiod remained at 16 h light/day throughout a previous study by Keshavarz and Quimby (2002) in which 10 mg T₄/kg was added to the feed. The photoperiod serves as the primary environmental signal that regulates reproductive function in many avian species. Increasing the photoperiod promotes maturation of the gonads and reproductive tract, whereas reducing the photoperiod causes the gonads and reproductive tract to regress and molting to occur. Reducing the photoperiod to ≦10 h/day during molting also tends to improve the post-molt performance of hens, presumably because the development of the ovaries and reproductive tract can be naturally photostimulated by gradually increasing the photoperiod as molted hens are brought back into lay (Berry, 2003). It is likely that photoperiod reduction will be used in commercial molting, either before (preconditioning), during, or after the molt treatment period, to permit response to post-molt photostimulation of the hens (DeCuypere and Verheyen, 1986; Hoshino et al., 1988; Biggs et al., 2003).

The experiment consisted of a 7-day acclimation period, 12 days of feeding the test diets, and 10 days of photoperiod adjustment (Reduced Daylength Group in Chamber 5 and Control group in Chamber 6). Reducing the photoperiod to 8 hours/day (0800 hours to 1600 hours) in Chamber 5 did not consistently reduce egg production or variability in egg production when compared with the initial 12 days for this group, or when compared with the Control group in Chamber 6 (17 hr light). Feeding 20 and 40 mg T₄/kg significantly reduced egg production within 4 days in Chamber 5 (8 hr light), and within 6 (40 mg T₄/kg) or 8 (20 mg T₄/kg) days in Chamber 6 (17 hr light). Only the hens fed the 40 mg T₄/kg diet in Chamber 6 (17 hr light) entirely ceased egg production for the remainder of the experiment whereas sporadic egg production continued by several hens in the other test diet groups. No mortality occurred in any of the Groups throughout the experiment. None of the hens in the Control groups or 20 mg T₄/kg groups molted in either chamber, 58% (7/12) of the hens in the 40 mg T₄/kg Group in Chamber 5 (8 hr light) molted fully (shed virtually all feathers within 7 to 10 days), and 100% of the hens in the 40 mg T₄/kg Group in Chamber 6 (17 hr light) molted fully. Feather regrowth subsequently progressed well in both 40 mg T₄/kg Groups, regardless of the ongoing difference in photoperiod. Behavioral changes were not apparent in molting hens, regardless of the test diet or Chamber. No cannibalism or aggression was noted within or between cages.

The Control Groups maintained or increased their body weight over the course of the experiment. All T₄ test diets caused reductions in body weight, with absolute body weight tending to return toward the initial values after cessation of feeding the test diets. Reduction in feed intake paralleled the respective contemporaneous changes in body weight. Thus, hens in Chamber 6 (17 hr light) that were fed the 40 mg T₄/kg test diet completely ceased egg production, shed virtually all of their feathers, reduced their feed intake by approximately 65%, and lost approximately 18% of their initial body weight. Hens in Chamber 5 (8 hr light) tended to have lower feed intake than hens in the respective Groups in Chamber 6 (17 hr light), presumably reflecting the impact of the reduced photoperiod (hours of light) on feed intake. Necropsies conducted at the end of experiment 3 revealed Group differences in ovary and oviduct weights that were consistent with contemporaneous egg production values. For example, the Control Groups in both chambers and the 20 mg T₄/kg Group in Chamber 6 (8 hr light) averaged between 50 and 60% hen-day egg production on day 34, and these groups also had the highest ovary and oviduct weights at the end of the experiment. In contrast, some hens in the 20 and 40 mg T₄/kg groups in Chamber 5 (8 hr light) continued to lay eggs sporadically, and all of the hens in the 40 mg T₄/kg group in Chamber 6 (17 hr light) ceased egg production entirely, as was reflected by proportional reductions in ovary and oviduct weights.

TABLE 8
Hen-day egg production (%) beginning on day 12 at 2-day intervals by treatment;
L-thyroxine administered in the diet at 0, 20, 40 mg/kg on day 13 for 12 days
with either 8 or 17 hour light (L) days by room (Experiment 3).

| Dose, mg/kg | Day | | | | | | | | | | | |
|---|---|---|---|---|---|---|---|---|---|---|---|---|
| of feed | 12 | 14 | 16 | 18 | 20 | 22 | 24 | 26 | 28 | 30 | 32 | 34 |
| 0 (8 hr L) | 85.4 | 60.4 | 79.2 | 81.3 | 56.3 | 72.9 | 56.3 | 93.8 | 70.8 | 64.6 | 54.2 | 58.3 |
| 20 (8 hr L) | 93.8 | 54.2 | 35.4 | 29.2 | 18.8 | 0.0 | 0.0 | 0.0 | 10.4 | 6.3 | 16.7 | 8.3 |
| 40 (8 hr L) | 83.3 | 66.7 | 16.7 | 8.3 | 12.5 | 4.2 | 8.3 | 4.2 | 4.2 | 8.3 | 4.2 | 8.3 |
| 0 (17 hr L) | 89.6 | 87.5 | 66.7 | 87.5 | 70.8 | 70.8 | 68.8 | 83.3 | 62.5 | 75.0 | 56.3 | 58.3 |
| 20 (17 hr L) | 91.7 | 58.3 | 35.4 | 31.3 | 14.6 | 27.1 | 4.2 | 18.8 | 0.0 | 29.2 | 41.7 | 54.2 |
| 40 (17 hr L) | 66.7 | 56.3 | 37.5 | 16.7 | 12.5 | 0.0 | 0.0 | 0.0 | 0.0 | 0.0 | 0.0 | 0.0 |

Note:
There were 4 cages of 3 hens each per treatment group.

TABLE 9
Body weight (BW) at day 12 (pre-molt), 22 (end of molt), and 34
(final) and body weight change (% BWC) from day 1 by thyroxine
treatments in 8 or 17 hour daily light (L) rooms (Experiment 3).

| Dose, mg/kg | Day 12 | Day 22 | | Day 34 | |
|---|---|---|---|---|---|
| of feed | BW, g | BW, g | % BWC | BW, g | % BWC |
| 0 (8 hr L) | 1443 | 1436 | −0.5 | 1487 | 3.0 |
| 20 (8 hr L) | 1492 | 1301 | −12.8 | 1332 | −10.7 |
| 40 (8 hr L) | 1488 | 1222 | −17.9 | 1300 | −12.6 |
| 0 (17 hr L) | 1514 | 1480 | −2.2 | 1534 | 1.3 |
| 20 (17 hr L) | 1572 | 1363 | −13.3 | 1453 | −7.6 |
| 40 (17 hr L) | 1436 | 1178 | −18.0 | 1224 | −14.8 |

Note:
Thyroxine treatment was added to diets on day 13 for 12 days.

TABLE 9
Feed consumption (FC, g/hen/day) and feed consumption change (% FCC)
from days 1-12, by thyroxine treatments in 8 or 17 hour daily light (L) rooms,
including days 13-15, 16-22, and 30-34 (Experiment 3).

| Dose, mg/kg | Days 1-12 | Days 13-15 | | Days 16-22 | | Day 30-34 | |
|---|---|---|---|---|---|---|---|
| of feed | FC, g/hen/d | FC, g/hen/d | % FCC | FC, g/hen/d | % FCC | FC, g/hen/d | % FCC |
| 0 (8 hr L) | 113.5 | 77.5 | −31.7 | 80.5 | −29.1 | 103.8 | −8.5 |
| 20 (8 hr L) | 121.3 | 41.8 | −65.5 | 44.5 | −63.3 | 82.5 | −32.0 |
| 40 (8 hr L) | 115.8 | 36.8 | −68.2 | 35.3 | −69.5 | 104.8 | −9.5 |
| 0 (17 hr L) | 110.0 | 94.0 | −14.5 | 103.0 | −6.4 | 138.5 | −25.9 |
| 20 (17 hr L) | 121.5 | 58.0 | −52.3 | 51.8 | −57.4 | 135.0 | 11.1 |
| 40 (17 hr L) | 103.3 | 42.0 | −59.3 | 37.5 | −63.7 | 93.3 | −9.7 |

Note:
Thyroxine treatment was added to diets on day 13 for 12 days.

TABLE 10
Ovary and oviduct weights per hen on day 34 as affected by
previous dietary thyroxine treatments in 8 or 17 hour daily
light (L) rooms (Experiment 3).

| Dose, mg/kg | Ovary Weight | | | Oviduct Weight | | |
|---|---|---|---|---|---|---|
| of feed | g | Std Dev | SEM | g | Std Dev | SEM |
| 0 (8 hr L) | 38.54 | 8.53 | 2.70 | 52.84 | 6.52 | 2.06 |
| 20 (8 hr L) | 7.91 | 13.98 | 4.42 | 18.64 | 21.97 | 6.94 |
| 40 (8 hr L) | 9.32 | 16.32 | 4.71 | 14.25 | 19.62 | 5.66 |
| 0 (17 hr L) | 45.99 | 8.16 | 2.46 | 59.95 | 7.45 | 2.25 |
| 20 (17 hr L) | 31.41 | 20.38 | 6.44 | 43.81 | 24.78 | 7.84 |
| 40 (17 hr L) | 3.82 | 2.86 | 0.86 | 8.03 | 3.08 | 0.93 |

Note:
The thyroxine treatment (molt) period was 10 days followed by 24 days on control feed, ending the study on day 34.
Std Dev is standard deviation, and
SEM is standard error of mean.

Experiment 4. Thyroactive Iodinated Casein Feeding Trial. Twenty of these HyLine W36 SCWL hens (60 wk old) were housed at one hen per cage in Chambers 5 and 6 of the Poultry Environmental Research Laboratory on the University of Arkansas Poultry Research Farm. The photoperiod was 18 hours/day and the temperature was 75° F. (24° C.) throughout this experiment. All cages were equipped with low-pressure nipple waterers and the hens were provided ad libitum a mash-type corn-soy-based layer diet formula-ted by the University of Arkansas Poultry Feed Mill. Daily egg production was recorded by cage for the duration of the experiment. Non-laying hens were culled during the acclimation period, leaving 14 active layers. Three of these hens remained on the Control feed throughout the experiment and, depending on the quantity of iodinated casein produced in batches 2 to 5, the remaining hens received feed blended with iodinated casein for 7 to 25 days (see below). Hens that had hot died before the end of the experiment the hens were euthanized with CO₂ gas.

Five batches of thyroactive iodinated casein were prepared using a “consensus” recipe based on methods described by Reineke and Turner (1942), Reineke et al. (1943), and Pitt-Rivers and Randall (1945). Batch #1 started with a pH that was too alkaline (>12), and the resulting material had a plastic-like consistency that solidified into an extremely hard and brittle mass. This batch was not fed to chickens. Batches 2 to 5 represented minor modifications using “KI” as an iodine source (Batches 2, 4, 5), or purified “I” as the iodine source (Batch 3). After each product was isolated, dried, and weighed, it then mixed at 1 part iodinated casein product to 2 parts (by weight) of standard laying hen diet. Feed mixed with batches 2 to 5 were fed to one or more hens.

The recipe that can be prepared in a 20-L plastic container shaped to fit into a laboratory water bath, and that can be used with confidence to molt SCWL hens is summarized as follows.

Consensus Recipe for Iodinated Casein
a. Mix 14 L of distilled H₂O with 3.325 g MnSO4.H₂O and 315 g of NaHCO3. Dissolve with stirring and bring the solution up to the water bath temperature of 39° C. The initial pH should be approximately 8.00.
b. With stirring (a length of copper tubing was used as a manual stirring rod throughout) blend in 945 g Casein (Erie Foods International, Inc., Erie IL 61250. Edible Casein, CAS#9000-71-9) without allowing clumps or foam to develop. The pH should be in the range of 7.00 to 7.20. [NOTE: This is 2× the proportion of casein:water used by the primary reference sources, but is designed to maximize the product produced in small volume lab batches.]
c. Stir in 173.25 g of “KI” (VWR Scientific No. VW5225-5; FW 166.00) or 132.3 g of “I” (E. M. Science No. IXO 126/2 Iodine USP, FW 126.90). Add the iodine source gradually with occasional stirring over the course of 2 hours. When dissolved, the pH should be between 7.20 and 7.30. Note: These proportions of added iodine represent approximately 14% I by weight of casein, as per the recommendations of Reineke et al. (1942, 1943).
d. Raise the temperature to 70° C. and incubate with occasional stirring for 20 hours. The pH should increase to >8.00 after 8 hours, and to ≧9.00 at the end of this 20 hour incubation.
e. Titrate with glacial acetic acid and continuous stirring until the protein flocculates and floats to the surface. Keep titrating with more acid and periodically scoop off the floating material and drain on cheese cloth or cotton cloth. When all the protein has been precipitated, pour the remaining liquid through cloth to harvest the dispersed material. Consolidate the material in one lump, wrap it in cloth, squeeze out all excess liquid, then break up the resulting cake into fine particles for drying.
f. Dry at 50° C. overnight in a forced-air oven. Periodically break up the material into very fine particles as it dries.

g. Weigh the dry product. Actual dry weight for Batch 5 using “KI” was 912.55 g. This was mixed with 1,825.1 g feed (laying hen diet), and run in 200 g batches through a Waring blender to mix. The 200 g batches then were blended together and remixed to achieve homogeneity.

TABLE 11
Egg production records for individual hens that remained on control feed (C; n = 3 hens)
or that received dietary thyroactive iodinated casein (TIC) from batches 2 (n = 1), 3 (n = 3),
4 (n = 2), and 5 (n = 5); number 1 indicates an egg was laid on that day (Experiment 4).

| Hen | 14 Days (Control Feed) | | | | | | | | | | | | | | TIC |
|---|---|---|---|---|---|---|---|---|---|---|---|---|---|---|---|
| No. | −14 | | −12 | | −10 | | −8 | | −6 | | −4 | | −2 | | Batch |
| 5 | 1 | 1 | 1 | 1 | 1 | 1 | 1 | 1 | 1 | 1 | 1 | 1 | 1 | | 5 |
| 6 | 1 | 1 | 1 | | 1 | | 1 | | 1 | 1 | 1 | 1 | 1 | 1 | 5 |
| 7 | 1 | 1 | 1 | 1 | 1 | 1 | 1 | 1 | 1 | 1 | 1 | | 1 | | 5 |
| 8 | | 1 | 1 | | 1 | 1 | 1 | | 1 | 1 | 1 | | | | C |
| 9 | 1 | 1 | 1 | 1 | 1 | 1 | 1 | 1 | 1 | 1 | | 1 | 1 | 1 | 5 |
| 10 | 1 | 1 | 1 | 1 | 1 | 1 | 1 | 1 | 1 | 1 | 1 | 1 | 1 | 1 | 5 |
| 11 | 1 | 1 | 1 | 1 | 1 | | 1 | 1 | 1 | 1 | 1 | 1 | 1 | | C |
| 12 | 1 | 1 | | | 1 | 1 | 1 | 1 | 1 | 1 | 1 | 1 | 1 | 1 | C |
| 14 | 1 | 1 | | | 1 | 1 | 1 | 1 | 1 | 1 | 1 | | | 1 | 3 |
| 15 | 1 | | 1 | 1 | 1 | 1 | 1 | 1 | 1 | 1 | 1 | 1 | 1 | 1 | 3 |
| 16 | 1 | 1 | 1 | 1 | 1 | 1 | 1 | 1 | 1 | 1 | 1 | 1 | 1 | 1 | 2 |
| 17 | 1 | | | 1 | | 1 | 1 | | 1 | 1 | 1 | 1 | | 1 | 4 |
| 18 | 1 | 1 | 1 | 1 | 1 | | | 1 | | 1 | 1 | 1 | | 1 | 3 |
| 21 | 1 | 1 | 1 | 1 | 1 | 1 | 1 | | | 1 | 1 | | | 1 | 4 |

| Hen | 18 Days of Thyroxine Treatment | | | | | | | | | | | | | | | | | |
|---|---|---|---|---|---|---|---|---|---|---|---|---|---|---|---|---|---|---|
| No. | 0 | | 2 | 4 | | 6 | | 8 | | 10 | | 12 | | 14 | | 16 | | 18 |
| 5 | 1 | 1 | 1 | | | | | | | | C | M | | | | | | E |
| 6 | 1 | 1 | | | | | | | | | | | | | | | M | E |
| 7 | | | | 1 | | | | | | | C | M | | | | | | E |
| 8 | | | | 1 | | 1 | 1 | | 1 | 1 | 1 | | 1 | 1 | 1 | 1 | 1 | E |
| 9 | | 1 | | 1 | | | | | | | C | | | M | | | | E |
| 10 | 1 | 1 | 1 | | 1 | | | C | | | | | M | | | | | E |
| 11 | 1 | 1 | 1 | | 1 | 1 | 1 | 1 | 1 | 1 | 1 | | 1 | 1 | 1 | 1 | 1 | E |
| 12 | 1 | 1 | 1 | | 1 | 1 | 1 | | 1 | 1 | 1 | 1 | 1 | 1 | 1 | 1 | 1 | E |
| 14 | | 1 | | | | | D | | | | | | | | | | | |
| 15 | | 1 | | | | | | | | | M | | | | | | | |
| 16 | | 1 | 1 | 1 | | | | | | | | M | C | | | | | |
| 17 | 1 | 1 | 1 | | | | | | | | | | | | M | | | |
| 18 | 1 | | | | | | | | M | | D | | | | | | | |
| 21 | 1 | 1 | | | | | | | | | | | | | M | | | |

Notes:
C is control feed;
M is molt feathers;
D is died; and
E is euphanized for necropsy.

Experiment 5. Molting Cobb Broiler Breeder Hens and Roosters with Dietary Thyroxine.

TABLE 12

| | | | | | 50 | | | 51 | | |
|---|---|---|---|---|---|---|---|---|---|---|
| | | 49 | | | Treatment 1 (25 ppm | | | Treatment 2 (40 ppm | | |
| 2004 | PEN | CONTROL GROUP | | | L-thyroxine) | | | L-thyroxine) | | |
| DATE | TRT | # Hens | # Roosters | # Eggs | # Hens | # Roosters | # Eggs | # Hens | # Roosters | # Eggs |
| 13-Sep | | 30 | 3 | 16 | 30 | 3 | 20 | 30 | 3 | 16 |
| 14-Sep | | 30 | 3 | 19 | 30 | 3 | 16 | 30 | 3 | 21 |
| 15-Sep | | 30 | 3 | 17 | 30 | 3 | 11 | 30 | 3 | 15 |
| 16-Sep | | 30 | 3 | 17 | 30 | 3 | 15 | 30 | 3 | 12 |
| 17-Sep | | 30 | 3 | 15 | 30 | 3 | 14 | 30 | 3 | 19 |
| 18-Sep | | 30 | 3 | 20 | 30 | 3 | 11 | 30 | 3 | 19 |
| 19-Sep | | 30 | 3 | 18 | 30 | 3 | 17 | 30 | 3 | 20 |
| 20-Sep | | 30 | 3 | 16 | 30 | 3 | 15 | 30 | 3 | 16 |
| 21-Sep | | 30 | 3 | 17 | 30 | 3 | 14 | 30 | 3 | 16 |
| 22-Sep* | | 30 | 3 | 18 | 30 | 3 | 14 | 30 | 3 | 18 |
| 23-Sep | | 30 | 3 | 16 | 30 | 3 | 19 | 30 | 3 | 15 |
| 24-Sep* | | 30 | 3 | 12 | 30 | 3 | 7 | 30 | 2 | 10 |
| 25-Sep | | 30 | 3 | 20 | 30 | 3 | 8 | 29 | 2 | 11 |
| 26-Sep | | 30 | 3 | 18 | 30 | 3 | 9 | 29 | 2 | 6 |
| 27-Sep | | 30 | 3 | 12 | 30 | 3 | 4 | 29 | 2 | 5 |
| 28-Sep | | 30 | 3 | 20 | 30 | 3 | 3 | 29 | 1 | 0 |
| 29-Sep | | 30 | 3 | 19 | 30 | 3 | 1 | 29 | 0 | 1 |
| 30-Sep | | 30 | 3 | 17 | 30 | 2 | 0 | 29 | 0 | 0 |
| 1-Oct | | 30 | 3 | 13 | 30 | 2 | 3 | 29 | 0 | 1 |
| 2-Oct | | 30 | 3 | 17 | 30 | 1 | 1 | 29 | 0 | 0 |
| 3-Oct | | 30 | 3 | 18 | 30 | 1 | 0 | 29 | 0 | 0 |
| 4-Oct | | 30 | 3 | 19 | 30 | 1 | 1 | 28 | 0 | 0 |
| 5-Oct | | 30 | 3 | 16 | 30 | 1 | 1 | 28 | 0 | 0 |
| 6-Oct | | 30 | 3 | 14 | 30 | 1 | 0 | 28 | 0 | 0 |
| 7-Oct | | 30 | 3 | 16 | 30 | 1 | 0 | 28 | 0 | 0 |
| 8-Oct* | | 30 | 3 | 12 | 27 | 0 | 3 | 25 | 0 | 0 |
| 9-Oct | | 30 | 3 | 17 | 27 | 0 | 1 | 25 | 0 | 0 |
| 10-Oct | | 30 | 3 | 12 | 26 | 0 | 0 | 25 | 0 | 0 |
| 11-Oct | | 30 | 3 | 16 | 26 | 0 | 0 | 25 | 0 | 0 |
| 12-Oct | | 30 | 3 | 13 | 26 | 0 | 0 | 25 | 0 | 0 |
| 13-Oct | | 30 | 3 | 17 | 26 | 0 | 0 | 25 | 0 | 0 |
| 14-Oct | | 30 | 3 | 15 | 26 | 0 | 0 | 25 | 0 | 0 |
| 15-Oct | | 30 | 3 | 15 | 25 | 0 | 0 | 25 | 0 | 0 |
| 16-Oct | | 30 | 3 | 12 | 25 | 0 | 0 | 25 | 0 | 0 |
| 17-Oct | | 30 | 3 | 17 | 24 | 0 | 0 | 25 | 0 | 0 |
| 18-Oct | | 30 | 3 | 18 | 24 | 0 | 0 | 25 | 0 | 0 |
| 19-Oct | | 30 | 3 | 9 | 23 | 0 | 0 | 25 | 0 | 0 |
| 20-Oct | | 30 | 3 | 16 | 23 | 0 | 0 | 25 | 0 | 0 |
| 21-Oct | | 30 | 3 | 19 | 23 | 0 | 0 | 25 | 0 | 0 |
| 22-Oct | | 30 | 3 | 10 | 22 | 0 | 0 | 25 | 0 | 0 |
| 23-Oct | | 30 | 3 | 14 | 22 | 0 | 0 | 25 | 0 | 0 |
| 24-Oct | | 30 | 3 | 13 | 21 | 0 | 0 | 25 | 0 | 0 |
| 25-Oct | | 30 | 3 | 20 | 21 | 0 | 0 | 25 | 0 | 0 |
| 26-Oct | | 30 | 3 | 13 | 21 | 0 | 0 | 25 | 0 | 0 |
| 27-Oct | | 30 | 3 | 16 | 21 | 0 | 0 | 25 | 0 | 0 |
| 28-Oct | | 30 | 3 | 12 | 21 | 0 | 0 | 25 | 0 | 0 |
| 29-Oct | | 30 | 3 | 17 | 21 | 0 | 0 | 25 | 0 | 3 |
| 30-Oct | | 30 | 3 | 12 | 21 | 0 | 0 | 25 | 0 | 3 |
| 31-Oct | | 30 | 3 | 13 | 21 | 0 | 0 | 25 | 0 | 3 |
| 1-Nov | | 30 | 3 | 17 | 21 | 0 | 0 | 25 | 0 | 1 |
| 2-Nov | | 30 | 3 | 15 | 21 | 0 | 0 | 25 | 0 | 3 |
| 3-Nov | | 30 | 3 | 13 | 21 | 0 | 0 | 25 | 0 | 3 |
| 4-Nov | | 30 | 3 | 15 | 21 | 0 | 0 | 25 | 0 | 5 |
| 5-Nov | | 30 | 3 | 13 | 21 | 0 | 0 | 24 | 0 | 5 |
| 6-Nov | | 30 | 3 | 11 | 21 | 0 | 0 | 24 | 0 | 4 |
| 7-Nov | | 30 | 3 | 18 | 21 | 0 | 0 | 24 | 0 | 4 |

*Notes:
September 22 -- placed on test feed.
September 24 -- accidental death of rooster treatment 2.
October 8 -- 3 hens sampled per treatment in treatments 1 and 2.

Cobb broiler breeder hens reduced their feed intake, ceased egg production, and began to molt feathers within about 15-17 days on thyroxine treated feed, a very similar but slightly delayed response compared to caged laying hens. Roosters began to “stroke blood” from the nostrils due to heat production and/or increased blood pressure associated with 25 or 40 mg/kg diet inclusion levels of L-thyroxine; therefore, males tolerate lower levels of L-thyroxine than hens apparently due to different hormonal makeups.

Experiment 6. Molting of Caged Laying with Dietary L-Thyroxine or Thyroactive Iodinated Casein Plus MgO or Sodium Salicylate.

TABLE 13
Bovans Caged Laying Hen Thyroxine Molting Trial Started May 25, 2005 (End of
First Cycle of Egg Production)

| | 25 May 05 | 9 Jun. 05 | Days to | | 4 Jun. 05 | 9 Jun. 05 |
|---|---|---|---|---|---|---|
| | Initial | (9th d Trt) | 0% Egg | 2&4 Jun. 05 | Shell + | Ovary + |
| Dietary | Body Wt, | Body Wt | Prod. (by | Egg | Membrane, | Oviduct, |
| Treatment¹ | lb | Change, lb | Replicate) | Weight, g | mm | % B Wt |
| Feed Removal | 3.60 | −0.79^{a} | 9.0^{b} | 59.78 | 0.395^{b} | 2.42 |
| 20 mg T₄/kg (L-T) | 3.49 | −0.50^{b} | 12.0^{a} | 58.68 | 0.466^{a} | 4.59 |
| 40 mg T₄/kg (L-T) | 3.57 | −0.60^{b} | 11.0^{a} | 60.17 | 0.446^{a} | 3.19 |
| 40 mg T₄/kg (TIC) | 3.51 | −0.54^{b} | 11.0^{a} | 58.46 | 0.424^{a} | 4.47 |
| 40 mg T₄/kg (TIC) + | 3.55 | −0.54^{b} | 12.0^{a} | 58.11 | 0.476^{a} | 3.35 |
| 1,500 mg Mg/kg | | | | | | |
| 40 mg T₄/kg (TIC) + | 3.58 | −0.62^{b} | 12.0^{a} | 59.95 | 0.432^{a} | 4.20 |
| 1,691 g SS/kg | | | | | | |
| P value | 0.35 | <0.001 | <0.001 | 0.23 | <0.001 | >0.43 |

¹T₄ is thyroxine; L-T is L-thyroxine; TIC is thyroactive iodinated casein; Mg supplied by MgO; and SS is sodium salicylate (460 g/quart solution) as Unisol ™ (Animal Science Products, Nacogdoches, TX). A 7-day pretest began May 25, followed by treatments, with 10 hours of light daily during pretest and treatments.

In Experiment 6, a conventional feed withdrawal molting procedure was compared with 5 dietary thyroxine treatments. Body weight loss after 9 days was greater, days to 0% egg production (9 days) shorter, ovary plus oviduct weight numerically lighter on day 9 of treatment, but eggs collected on day 4 of treatment had thinner shells, in the feed withdrawal group. Thyroactive iodinated casein (TIC) was as effective as L-thyroxine (11 days to 0% egg production and −0.54 lb weight loss each) when contributing 40 mg T4/kg diet. The Mg added to TIC numerically increased shell plus membrane thickness (0.476 mm with Mg and 0.424 mm unsupplemented), and sodium salicylate (SS) added to TIC numerically increased weight loss (−0.62 lb with SS and −0.54 lb unsupplemented). The 10-hour light days during the 7-day pretest and the molting treatment period was evaluated to hasten the cessation of egg production, but unfortunately it appeared to be counterproductive probably due to reduced treated feed intake on the shorter day length.

Following are recent assays of thyroactive iodinated casein (1% thyroxine), manufactured in a foreign country, and used in Experiments 6 and 7. Assays were conducted at a commercial lab in the U.S. on Sep. 13, 2004 using enzymatic hydrolysis and HPLC.

TABLE 1
Assay of thyroactive iodinated casein (˜1% thyroxine activity) by HPLC.

| | (MIT; T₁) | (DIT; T₂) | (T_{1;} T₂) | (T₃) | | “Iodotyrosines” |
|---|---|---|---|---|---|---|
| | Monoiodo- | Diiodo- | Mono-&Diiodo- | Triiodo- | (T₄) | Combined |
| | tyrosine | tyrosine | thyronines | thyronine | Thyroxine | Total |
| | (%) | (%) | (%) | (%) | (%) | (%) |
| Lot# 1 | 1.39 | 2.76 | 0.21 | 0.37 | 0.95 | 5.68 |
| Lot# 2 | 1.49 | 3.11 | 0.30 | 0.42 | 0.97 | 6.29 |
| Lot# 3 | 1.22 | 2.46 | 0.20 | 0.35 | 0.92 | 5.15 |
| Lot# 4 | 1.20 | 2.64 | 0.17 | 0.37 | 0.78 | 5.16 |
| Average | 1.33 | 2.74 | 0.22 | 0.38 | 0.91 | 5.57 |

The thyroactive iodinated casein, also known as thyroprotein, had a combination of iodine compounds indicating partial iodination of tyrosine during the process. The product had an overall average content of 0.91% thyroxine based on assay of samples from 4 lots.

Experiment 7. Molting of Turkey Breeder Hens with Dietary L-Thyroxine, Porcine Thyroid Powder, or Thyroactive Iodinated Casein With or Without Protease. Turkey breeder hens were molted with various dietary thyroxine treatments at Diamond K Research, Marshville, N.C. (Jun. 20-Jul. 1, 2005). Table 13 contains the necropsy results at the end of the 10-day molting treatment period.

TABLE 13
Effect of dietary thyroxinic compounds fed for 10 days on turkey breeder
hen body weight and weights of ovary, oviduct, and liver (Experiment 7).

| | Ending | | | |
|---|---|---|---|---|
| Dietary | (10 d) | Ovary | Oviduct | Liver |
| Treatment | Body Wt, lb | Weight, g | Weight, g | Weight, g |
| Feed & Water | 23.67 | 72.7^{b} | 70.0^{b} | 171.8^{a} |
| Restriction | | | | |
| Control Feed | 24.07 | 165.2^{a} | 119.6^{a} | 182.0^{a} |
| (ad libitum) | | | | |
| 10 mg T₄/kg (L-T) | 23.03 | 159.1^{a} | 124.1^{a} | 143.0^{bc} |
| 25 mg T₄/kg (L-T) | 25.11 | 160.5^{a} | 134.7^{a} | 144.6^{b} |
| 40 mg T₄/kg (L-T) | 22.99 | 129.1^{a} | 138.7^{a} | 141.9^{bc} |
| 40 mg T₄/kg (TIC) | 23.24 | 132.5^{a} | 133.2^{a} | 142.6^{bc} |
| 40 mg T₄/kg (TIC) + | 23.95 | 125.8^{a} | 121.3^{a} | 134.0^{bc} |
| Protease | | | | |
| 40 mg T₄/kg (PTP) | 23.15 | 42.1^{b} | 69.6^{b} | 123.1^{c} |
| P value | 0.277 | <0.001 | <0.001 | <0.001 |

¹T₄ is thyroxine; L-T is L-thyroxine; TIC is thyroactive iodinated casein; Protease is Versazyme ™ (BioResources International, Inc., Raleigh, NC) at 0.10% of diet; and PTP is defatted, desiccated porcine thyroid powder. There was a 3-day pretest acclimation period after transporting the turkey hens to the research site. There were 6 individually penned hens (on litter) per treatment.

At 40 mg T₄ kg diet, porcine thyroid powder was most effective. The thyroactive iodinated casein alone (40 mg T₄/kg diet) or Sigma L-thyroxine (10, 20, or 40 mg T₄/kg diet) were not as effective as porcine thyroid powder at regressing reproductive tracts. Adding protease was without effect. No feather molt occurred in any treatment during the 10-day molting treatment period.

Although the present invention has been described in the context of compositions, examples, methods, preferred embodiments, procedures, and processes to illustrate further practice of the invention, it will be readily apparent to those skilled in the art that numerous modifications and variations can be made therein without departing from the spirit or scope of the invention. Also, the appended claims of the present invention may be practiced otherwise than as particularly described. It is intended that the above description be interpreted as illustrative, and not in a limiting sense.

1. A method for safely improving a property of a product produced by an animal, which property it is desired to improve according to a desired improvement, said method comprising administering to the animal an effective amount of a thyroxinic substance.

2. The method of claim 1, wherein the property is shell thickness, wherein the desired improvement is an increase, and wherein the product is an egg.

3. The method of claim 1, wherein the property is iodine content, wherein the desired improvement is an increase, and wherein the product is milk or semen or an egg.

4. The method of claim 1, wherein the property is growth rate, wherein the desired improvement is an increase, and wherein the product is integument.

5. The method of claim 1, wherein the property is quantity, wherein the desired improvement is an increase, and wherein the product is milk.

6. A method for safely improving a property of an animal or of a tissue or an organ or a fluid of an animal, which property it is desired to improve according to a desired improvement, said method comprising administering to the animal an effective amount of a thyroxinic substance or a goitrogenic substance.

7. The method of claim 6, wherein the property is leanness, wherein the desired improvement is an increase, wherein the tissue is muscle tissue, and wherein the administering comprises administering an effective amount of a thyroxinic substance.

8. The method of claim 6, wherein the property is iodine content, wherein the desired improvement is an increase, wherein the tissue is blood or muscle tissue, and wherein the administering comprises administering an effective amount of a thyroxinic substance.

9. The method of claim 6, wherein the property is strength, wherein the desired improvement is an increase, wherein the tissue is bone, and wherein the administering comprises administering an effective amount of a thyroxinic substance.

10. The method of claim 6, wherein the property is size, wherein the desired improvement is an increase, wherein the organ is a testis, and wherein the administering comprises administering an effective amount of a goitrogenic substance.

11. The method of claim 6, wherein the property is the proportion of total body weight of the animal made up of adipose tissue, wherein the desired improvement is a decrease, wherein the tissue is adipose tissue, and wherein the administering comprises administering an effective amount of a thyroxinic substance.

12. The method of claim 6, wherein the property is the weight of the animal, wherein the desired improvement is a decrease, and wherein the administering comprises administering an effective amount of a thyroxinic substance.

13. The method of claim 6, wherein the property is the basal metabolic rate of the animal, wherein the desired improvement is an increase, and wherein the administering comprises administering an effective amount of a thyroxinic substance.

14. The method of claim 6, wherein the property is the concentration of thyroxine, wherein the fluid is serum or plasma, wherein the desired improvement is an increase, and wherein the administering comprises administering an effective amount of a thyroxinic substance.

15. A method for safely altering a reproductive behavior of an animal, which reproductive behavior it is desired to alter according to a desired alteration, comprising administering to the animal an effective amount of a thyroxinic substance.

16. The method of claim 15, wherein the desired alteration is an increase of libido or fertility or both libido and fertility.

17. The method of claim 16, wherein the animal is a male poultry breeder.

18. The method of claim 15, wherein the alteration is an induction of reproductive quiescence.

19. The method of claim 18, wherein the animal is a wild pest bird or a poultry bird.

20. The method of claim 1, wherein the thyroxinic substance comprises iodinated casein, wherein the total L-thyroxine content of said iodinated casein exceeds 1%.

21. The method of claim 6, wherein the thyroxinic substance comprises iodinated casein, wherein the total L-thyroxine content of said iodinated casein exceeds 1%.

22. The method of claim 15, wherein the thyroxinic substance comprises iodinated casein, wherein the total L-thyroxine content of said iodinated casein exceeds 1%.
